(19) United States
(12) Patent Application Publication
(10) Pub. No.: US 2025/0134696 A1
(43) Pub. Date: May 1, 2025

(54) FLUID COLLECTION ASSEMBLIES INCLUDING A POROUS MATERIAL INCLUDING INNER AND OUTER LAYERS

(71) Applicant: PUREWICK CORPORATION, Covington, GA (US)

(72) Inventor: Shahab Siddiqui, Lawrenceville, GA (US)

(21) Appl. No.: 18/835,444

(22) PCT Filed: Feb. 7, 2022
(86) PCT No.: PCT/US2022/015420

§ 371 Date: Aug. 2, 2024

(51) Int. Cl. A61F 5/451; A61M 1/00

(52) U.S. Cl. CPC A61F 5/451; A61M 1/80; A61M 2205/02

(57) ABSTRACT

An example fluid collection assembly includes a fluid impermeable barrier. The fluid impermeable barrier at least defines a chamber, at least one opening, and a fluid outlet. The fluid collection assembly also includes at least one porous material disposed in the chamber. The porous material includes at least one outer porous material. The outer porous material facilitates the porous material receiving bodily fluids and having the bodily fluids flow therein.

### BACKGROUND

A person or animal may have limited or impaired mobility so typical urination processes are challenging or impossible. For example, a person may experience or have a disability that impairs mobility. A person may have restricted travel conditions such as those experienced by pilots, drivers, and workers in hazardous areas. Additionally, sometimes bodily fluids collection is needed for monitoring purposes or clinical testing.

Urinary catheters, such as a Foley catheter, can address some of these circumstances, such as incontinence. Unfortunately, urinary catheters can be uncomfortable, painful, and can lead to complications, such as infections. Additionally, bed pans, which are receptacles used for the toileting of bedridden individuals are sometimes used. However, bedpans can be prone to discomfort, spills, and other hygiene issues.

### SUMMARY

Embodiments are directed to fluid collection assemblies including at least one outer porous material, fluid collection systems including the same, and methods of forming and using the same. In an embodiment, a fluid collection assembly is disclosed. The fluid collection assembly includes a fluid impermeable barrier at least defining a chamber, at least one opening, and a fluid outlet. The fluid collection assembly also includes at least one porous material disposed in the chamber. The at least one porous material includes an outer layer and an inner layer. The outer layer includes at least one of a bamboo, cellulose, hydrophilic polypropylene, hydrophilic polyethylene, or hydrophilic polyester.

In an embodiment, a fluid collection system is disclosed. The fluid collection system includes a fluid collection assembly. The fluid collection assembly includes a fluid impermeable barrier at least defining a chamber, at least one opening, and a fluid outlet. The fluid collection assembly also includes at least one porous material disposed in the chamber. The at least one porous material includes an outer layer and an inner layer. The outer layer includes at least one of bamboo, cellulose, hydrophilic polypropylene, hydrophilic polyethylene, or hydrophilic polyester. The fluid collection system also includes a fluid storage container and a vacuum source. The chamber of the fluid collection assembly, the fluid storage container, and the vacuum source are in fluid communication with each that, when one or more bodily fluids are present in the chamber, a suction provided from the vacuum source to the chamber of the fluid collection assembly removes the one or more bodily fluids from the chamber and deposits the bodily fluids in the fluid storage container.

In an embodiment, a method of using a fluid collection system is disclosed. The method includes positioning a fluid collection assembly such that at least one opening defined by a fluid impermeable barrier of the fluid collection assembly is positioned adjacent to or receives a urethral opening. The fluid impermeable barrier of the fluid collection assembly at least defines a chamber and a fluid outlet. The fluid collection assembly includes at least one porous material disposed in the chamber. The at least one porous material includes an outer layer and an inner layer. The outer layer includes at least one of a bamboo, cellulose, hydrophilic polypropylene, hydrophilic polyethylene, or hydrophilic polyester.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a fluid collection assembly, according to an embodiment.

FIGS. 1B and 1C are cross-sectional schematics of the fluid collection assembly taken along planes 1B-1B and 1C-1C, respectively, shown in FIG. 1A.

FIG. 2A is an isometric view of a fluid collection assembly, according to an embodiment.

FIG. 2B is a cross-sectional schematic of the fluid collection assembly taken along plane 2B-2B shown in FIG. 2A.

FIG. 3 is a cross-sectional schematic of male fluid collection assembly, according to an embodiment.

FIG. 4 is a cross-sectional schematic of a fluid collection assembly, according to an embodiment.

FIG. 5 is a block diagram of a fluid collection system for fluid collection, according to an embodiment.

### DETAILED DESCRIPTION

Embodiments are directed to fluid collection assemblies including at least one of bamboo, cellulose, hydrophilic polypropylene, hydrophilic polyethylene, or hydrophilic polyester, fluid collection systems including the same, and methods of forming and using the same. An example fluid collection assembly includes a fluid impermeable barrier. The fluid impermeable barrier at least defines a chamber, at least one opening, and a fluid outlet. The fluid collection assembly also includes at least one porous material disposed in the chamber. The porous material includes at least one inner layer and at least one outer layer. The outer layer includes at least one outer porous material. As used herein, the outer porous material refers to at least one of bamboo, cellulose, hydrophilic polypropylene, hydrophilic polyethylene, or hydrophilic polyester.

During use, the fluid collection assembly may be positioned on an individual such that the opening is positioned adjacent to a female urethral opening or receives a male urethral opening (i.e., penis). The individual may discharge one or more bodily fluids, such as urine, blood, or sweat. The bodily fluids may flow through the opening and into the porous material. The bodily fluids may be removed from the chamber via the fluid outlet. In an embodiment, a suction may be applied to the chamber from a vacuum source which removes the bodily fluids from the chamber.

Some conventional fluid collection assemblies include a hydrophobic porous material. Examples of the hydrophobic porous material that may be used in conventional fluid collection assemblies include hydrophobic polyester foam, hydrophobic polyester fabric, hydrophobic polyester compression bandages, spandex compression bandages, polyamide compression bandages, hydrophobic polypropylene foams, hydrophobic polypropylene fabrics, spun nylon fibers, or other synthetic materials. The hydrophobic porous material exhibits a porosity and fluid permeability that allows the hydrophobic porous material to receive bodily fluids from an individual and have the bodily fluids flow therethrough. The hydrophobic porous material is also, inherently, hydrophobic (e.g., exhibits a contact angle with water that is greater than 90°). The hydrophobicity of the hydrophobic porous material pushes the bodily fluids received by the hydrophobic porous material towards an outlet of the conventional fluid collection assemblies and inhibits bodily fluids remaining in the hydrophobic porous material. As such, the hydrophobicity of the hydrophobic porous material allows the conventional fluid collection assembly to be relatively dry a short period of time after receiving one or more bodily fluids from the individual which prevents skin irritation and degradation. However, it has been found that the hydrophobic porous material exhibits relatively poor water absorption and wicking (i.e., spontaneous flow of the bodily fluids received thereby driven by capillary forces and/or capillary pressure). In particular, the hydrophobic porous material exhibits relatively poor water absorption and wicking since, at least initially, the hydrophobicity of the hydrophobic porous material resists receiving the bodily fluids and resists wetting the hydrophobic material. The relatively poor water absorption and wicking ability of the hydrophobic porous material may result in bodily fluids initially not being effectively received into the hydrophobic porous material which, in turn, causes the bodily fluids to leak from the conventional fluid collection assembly. The relatively poor wicking of the hydrophobic porous material may limit flow of the bodily fluids therethrough until the bodily fluids wet a significant portion of the surfaces of the hydrophobic porous material which may result, at least initially, in limited flow of the bodily fluids through the hydrophobic material and localized saturation of the bodily fluids in the hydrophobic porous material. The limited flow of the bodily fluids through the hydrophobic porous material and/or the localized saturation of the bodily fluids may also result in leakage of the bodily fluids from the hydrophobic porous material.

Some other conventional fluid collection assemblies attempt to resolve these issues associated with hydrophobic porous material by including a hydrophilic porous material. Example of hydrophilic porous materials that are used in conventional fluid collection assemblies include cotton and rayon formed from sources other than bamboo.

The hydrophilic porous material of the conventional fluid collection assemblies exhibit relatively good water absorption and wicking. However, unlike the hydrophobic porous materials discussed above, the hydrophilic porous material retains a significant quantity of bodily fluids received thereby and remains wet for a prolonged period of time. Since the hydrophilic porous material remains wet, the conventional fluid collection assemblies including the hydrophilic porous material may only be used for a short period of time after receiving the bodily fluids to prevent skin irritation and degradation.

Other conventional fluid collection assemblies attempt to resolve these issues associated with hydrophobic and hydrophilic porous materials by not including any porous materials or by spacing the porous materials from the skin of the individual. However, the conventional fluid collection assemblies that do not include any porous material or include the porous materials spaced from the skin have difficulty receiving bodily fluids and may result in the bodily fluids pooling, both of which may result in leakage of the bodily fluids, skin degradation, and skin irritation.

The conventional fluid collection assemblies discussed above may be external fluid collection assemblies. The external fluid collection assemblies are fluid collection assemblies that are not positioned into the urethral tube of the individual. The external fluid collection assemblies significantly reduce the risk of catheter-assisted urinary tract infections (“CAUTI”) compared to internal fluid collection assemblies (e.g., a Foley catheter). In an embodiment, the conventional external fluid collection assemblies are sterilized during the manufacturing and packaging process. Sterilizing the conventional external fluid collection assemblies during manufacturing and packaging process may be difficult and time consuming, especially when the conventional external fluid collection assemblies include a porous material. In an embodiment, the conventional external fluid collection assemblies are not sterilized during the manufacturing and packaging process. Not sterilizing the conventional external fluid collection assemblies makes the manufacturing and packaging of such external fluid collection assemblies much easier and quicker than if the conventional external fluid collection assemblies were sterilized. The unsterilized conventional external fluid collection assemblies are significantly less likely to cause CAUTI compared to conventional internal fluid collection assemblies. However, the unsterilized conventional external fluid collection assemblies are more likely to cause CAUTI compared to sterilized conventional external fluid collection assemblies. Also, both the sterilized and the unsterilized conventional external fluid collection assemblies are vulnerable to attack by microbials that may cause CAUTI during use of such fluid collection assemblies are used.

The fluid collection assemblies disclosed herein include at least one outer porous material. In other words, the fluid collection assemblies disclosed herein include at least one of bamboo, cellulose, hydrophilic polypropylene, hydrophilic polyethylene, or hydrophilic polyester. The outer porous material allows the fluid collection assemblies disclosed herein to be improvements over the conventional fluid collection assemblies. For example, similar to the hydrophobic porous materials used in conventional fluid collection assemblies, the outer porous material exhibits a porosity and fluid permeability that allows the outer porous material to receive and have the bodily fluids flow therein. However, unlike the hydrophobic porous materials, the outer porous material is hydrophilic, which allows the outer porous material to exhibit relatively good water absorption and wicking. The relatively good water absorption and wicking of the outer porous material decreases the likelihood that the outer porous material leaks. Also, it has been surprising found that the outer porous material does not exhibit the issues discussed above that are associated with other hydrophilic porous materials used in conventional fluid collection assemblies. For example, it has been found that the outer porous material does not retain a significant quantity of bodily fluids therein. As such, the outer porous material may be relatively dry a short period of time after the outer porous material receives the bodily fluids. Thus, the outer porous material exhibits many of the same benefits as the hydrophobic and hydrophilic porous materials without exhibiting many of the disadvantages of the hydrophobic and hydrophilic porous materials.

The outer porous material exhibit several properties other than good fluid permeability, good water absorption, good wicking, and not retaining significant quantities of bodily fluids therein. In an example, it has been found that the outer porous material exhibits what is described as a “soft feel” which enables the outer porous material to make the fluid collection assemblies including the outer porous material more comfortable to use than fluid collection assemblies including other materials. In an example, it has been found that the outer porous material exhibits better air flow conditions compared to other conventional porous materials which facilitates drying of the outer porous material. The outer porous materials are also able to be used in rapid high volume manufacturing processes, as will be discussed in more detail below.

In an embodiment, the outer porous material may include one or more chemicals that make the outer porous material an improvement over conventional porous materials. For example, when the outer porous material includes bamboo, the outer porous material may include bamboo kun, a material naturally found in bamboo. The bamboo kun causes the outer porous material be exhibit antifungal properties and antibacterial properties against both Gram-positive and Gram-negative bacteria. As such, the presence of the bamboo kun in the outer porous material causes the fluid collection assemblies to be substantially as likely to cause CAUTI as sterilized conventional external fluid collection assemblies without actually sterilizing the fluid collection assemblies. Also, the bamboo kun of the outer porous material is not vulnerable to attack by infectious microbials that may cause CAUTI. The bamboo kun also causes the outer porous material to be odor resistant, unlike porous materials used in conventional fluid collection assemblies. The odor resistant abilities of the outer porous material makes using the fluid collection assemblies including the outer porous material less embarrassing to use since, unlike conventional fluid collection assemblies, the fluid collection assemblies including the outer porous material are unlikely to have a noticeable odor of urine or blood. Unlike the porous materials of conventional fluid collection assemblies, the bamboo kun also causes the outer porous material to repel dust mites, other bugs, other infectious microorganisms, and viruses. The bamboo kun also repeals these organisms while the bamboo that forms the outer porous material is grown. As such, unlike the materials used to form other natural porous materials (e.g., cotton and cellulose), the bamboo that forms the outer porous material may be grown without pesticides, fungicides, and insecticides. This results in the outer porous material to be less likely to be contaminated with pesticides, fungicides, and insecticides compared to other natural porous materials without having to process the outer porous material to remove such materials. Also, the bamboo kun causes the outer porous material to be more hypoallergenic than other porous materials used in conventional fluid collection assemblies.

Bamboo is also very comfortable against the sensitive regions that are about the urethral opening of the individual (e.g., the penis and the vaginal region). For example, bamboo is able to receive and remove the bodily fluids while also being substantially dry a short period of time after receiving the bodily fluids. The bamboo is also a better conductor of heat than some porous materials used in conventional fluid collection assemblies which, combined with the breathability of the outer porous material, decreases sweat generated by and heat retained by the region about the urethral opening of the individual. Bamboo is also light and soft to the touch. Further, bamboo is also wrinkle-resistant which prevents the formation of prominent bumps on the outer porous material which may cause discomfort. Bamboo is also very clean compared to other porous materials, even after receiving bodily fluids, due to the antibacterial, antifungal, etc. properties thereof, as discussed above. Unlike at least some other porous materials used in conventional fluid collection assemblies, the outer layer including bamboo is also eco-friendly and is stain resistant.

As discussed above, the outer porous material exhibits the benefits of the hydrophobic and hydrophilic porous materials. However, the outer porous material also exhibits several benefits that neither or at least most of the hydrophobic and hydrophilic porous materials do not exhibit without difficult, costly, and time consuming manufacturing processes.

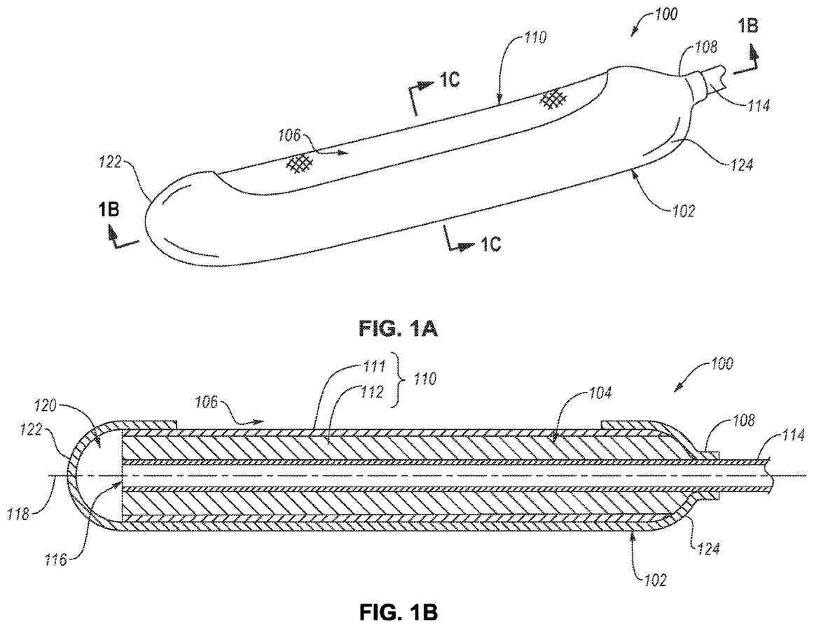

FIG. 1A is an isometric view of a fluid collection assembly 100, according to an embodiment. FIGS. 1B and 1C are cross-sectional schematics of the fluid collection assembly 100 taken along planes 1B-1B and 1C-1C, respectively, shown in FIG. 1A. The fluid collection assembly is an example of a fluid collection assembly configured to receive bodily fluids from a female urethral opening. The fluid collection assembly 100 includes a fluid impermeable barrier 102. The fluid impermeable barrier 102 at least defines a chamber 104, at least one opening 106, and a fluid outlet 108. The fluid collection assembly 100 also includes at least one porous material 110 disposed in the chamber 104 that extends across the opening 106. The porous material 110 includes an outer layer 111 and an inner layer 112. In an embodiment, the outer layer 111 includes the at least one outer porous material. That is, the outer layer 111 includes at least one of bamboo, cellulose, hydrophilic polypropylene, hydrophilic polyethylene, or hydrophilic polyester.

The fluid impermeable barrier 102 at least partially defines a chamber 104 (e.g., interior region) and an opening 106. The fluid impermeable barrier 102 temporarily stores the bodily fluids in the chamber 104. The fluid impermeable barrier 102 may be formed of any suitable fluid impermeable material(s), such as a fluid impermeable polymer (e.g., silicone, polypropylene, polyethylene, polyethylene terephthalate, neoprene, a polycarbonate, etc.), a metal film, natural rubber, another suitable material, any other fluid impermeable material disclosed herein, or combinations thereof. As such, the fluid impermeable barrier 102 substantially prevents the bodily fluids from passing through the fluid impermeable barrier 102. In an example, the fluid impermeable barrier 102 may be air permeable and fluid impermeable. In such an example, the fluid impermeable barrier 102 may be formed of a hydrophobic material that defines a plurality of pores. At least one or more portions of at least an outer surface of the fluid impermeable barrier 102 may be formed from a soft and/or smooth material, thereby reducing chaffing.

The opening 106 provides an ingress route for bodily fluids to enter the chamber 104. The opening 106 may be defined by the fluid impermeable barrier 102 such as by an inner edge of the fluid impermeable barrier 102. For example, the opening 106 is formed in and extends through the fluid impermeable barrier 102 thereby enabling bodily fluids to enter the chamber 104 from outside of the fluid collection assembly 100.

In some examples, the fluid impermeable barrier 102 may define a fluid outlet 108 sized to receive the conduit 114. The at least one conduit 114 may be disposed in the chamber 104 via the fluid outlet 108. The fluid outlet 108 may be sized and shaped to form an at least substantially fluid tight seal against the conduit 114 or the at least one tube thereby substantially preventing the bodily fluids from escaping the chamber 104.

As previously discussed, the fluid collection assembly 100 includes porous material 110 disposed in the chamber 104. The porous material 110 may cover at least a portion (e.g., all) of the opening 106. The porous material 110 is exposed to the environment outside of the chamber 104 through the opening 106. The porous material 110 may include an outer layer 111 and an inner layer 112 that is distinct from the outer layer 111.

The outer layer 111 of the porous material 110 is positioned within the chamber 104 to extend across the opening 106. The outer layer 111 is positioned in the porous material 110 to be closer to the urethral opening of the individual than the inner layer 112. In other words, the outer layer 111 includes the portions of the porous material 110 that initially receive the bodily fluids from the individual. As such, in an embodiment, the outer layer 111 may include the outer porous material. For example, the outer layer 111 is configured to quickly receive the bodily fluids discharged from the individual since the outer layer 111 initially receives the bodily fluids to prevent the bodily fluids leaking from the porous material 110. As previously discussed, the outer porous material is able to quickly receive the bodily fluids and, thus, forming the outer layer 111 from the outer porous material allows the outer layer 111 to quickly receive the bodily fluids.

In an embodiment, the outer porous material may be formed from any bamboo material. In an example, the outer porous material is formed from natural bamboo. The natural bamboo may be more ecologically friendly than other bamboo materials and may requires less manufacturing than non-natural bamboo. In an example, the outer porous material may include black bamboo (i.e., bamboo from Phyllostachys nigra). Black bamboo exhibits greater antimicrobial properties than other types of bamboo though, it is noted, the other types of bamboo also exhibit antimicrobial properties. In an example, the outer porous material includes rayon formed from bamboo. In an embodiment, the outer porous material may be formed from any cellulose material. In an example, the outer porous material is formed from naturally derived cellulose which may be more ecologically friendly than other cellulose materials. In an embodiment, the outer porous material is formed from at least one of hydrophilic polypropylene, hydrophilic polyethylene, or hydrophilic polyester. Generally, polypropylene, polyethylene, and polyester are hydrophobic materials. As such, the outer porous material may include at least one of modified polypropylene, modified polyethylene, or modified polyester that has been modified to increase the hydrophilicity (e.g., decrease the contact angle with water) thereof. For example, the polypropylene, polyethylene, and/or polyester may be modified using heat (e.g., flame), plasma treatment, chemical adhesion promoters (e.g., solvents such as toluene), smoothing the surface of the material, any other suitable modification, or combinations thereof. In an embodiment, the outer porous material may be formed from two or more of bamboo, cellulose, hydrophilic polypropylene, hydrophilic polyethylene, or hydrophilic polyester. In such an embodiment, for instance, the outer porous material may include bamboo such that the outer porous material include bamboo kun and a more readily available material, such as cellulose, hydrophilic polypropylene, hydrophilic polyethylene, or hydrophilic polyester thereby allowing the outer porous material to exhibit the antimicrobial properties of bamboo while inhibiting logistical issues associated with using a less readily available material.

In an embodiment, the outer porous material may by hydrophilic. The hydrophilicity of the outer porous material may cause the outer porous material to quickly receive bodily fluids therein thereby preventing or at least inhibiting leakage of bodily fluids caused by a large discharge of bodily fluids over a short period of time. The outer porous material may be hydrophilic when the outer porous material exhibits a contact angle with water (a major constituent of bodily fluids) that is about 0° to about 10°, about 5° to about 15°, about 10° to about 20°, about 15° to about 25°, about 20° to about 30°, about 25° to about 35°, about 30° to about 40°, about 35° to about 45°, about 40° to about 50°, about 45° to about 55°, about 50° to about 60°, about 55° to about 65°, about 60° to about 70°, about 65° to about 75°, about 70° to about 80°, about 75° to about 85°, or about 80° to 90°. Generally, increasing the hydrophilicity of the outer porous material (i.e., decreasing the contact angle between the outer porous material and water) increases the quantity of bodily fluids that the outer porous material may receive over a certain period of time. However, increasing the hydrophilicity of the outer porous material may increase the quantity of bodily fluids that are retained in the outer porous material after the outer porous material receives the bodily fluids. As such, the hydrophilicity of the outer porous material may be selected based on balancing the need to receive bodily fluids quickly while also keeping the porous material 110 dry. For example, a fluid collection assembly 100 configured to be used with an individual with a large bladder for short periods of time may include an outer porous material exhibiting a hydrophilicity that is greater than an outer porous material of a fluid collection assembly 100 configured to be used with an individual with an average to small sized bladder for long period of time.

In an embodiment, the hydrophilicity of the outer porous material may be an inherent property of the bamboo or cellulose fibers used to form the outer porous material. In an embodiment, the hydrophilicity of the outer porous material may be changed (e.g., increased or decreased) by at least one of impurities or functional groups added to the outer porous material, otherwise treating the outer porous material, or coating the outer porous material with a material that exhibits a hydrophilicity that is different than the outer porous material. For example, the hydrophilicity of at least one of the bamboo, cellulose, hydrophilic polypropylene, hydrophilic polyethylene, or hydrophilic polyester may be changed. It is noted that the hydrophilicity of the outer porous material may depend on the temperature, humidity, and other factors.

The outer porous material of the porous material 110 may be selected to exhibit a density of about 50 kg/m³ to about 100 kg/m³, about 75 kg/m³ to about 125 kg/m³, about 100 kg/m³ to about 150 kg/m³, about 125 kg/m³ to about 175 kg/m³, about 150 kg/m³ to about 200 kg/m³, about 175 kg/m³ to about 225 kg/m³, about 200 kg/m³ to about 250 kg/m³, about 225 kg/m³ to about 275 kg/m³, about 250 kg/m³ to about 300 kg/m³, about 275 kg/m³ to about 325 kg/m³, about 300 kg/m³ to about 350 kg/m³, about 325 kg/m³ to about 375 kg/m³, about 350 kg/m³ to about 400 kg/m³, about 375 kg/m³ to about 425 kg/m^{3,} about 400 kg/m³ to about 450 kg/m³, about 425 kg/m³ to about 475 kg/m³, about 450 kg/m³ to about 500 kg/m³, about 475 kg/m³ to about 525 kg/m³, about 500 kg/m³ to about 550 kg/m³, about 525 kg/m³ to about 575 kg/m³, or about 550 kg/m³ to about 600 kg/m³.

As previously discussed, the outer porous material may be formed from a hydrophilic material which may cause the outer porous material to retain the bodily fluids therein. To decrease the quantity of bodily fluids retained by the outer porous material, the outer layer 111 may be configured to be relatively thin. For example, the outer layer 111 may be configured to exhibit a thickness measured perpendicularly to the longitudinal axis 118 (e.g., measured radially) that is about 2 mm or less, about 1.5 mm or less, about 1.25 mm or less, about 1 mm or less, about 800 μm or less, about 700 μm or less, about 600 μm or less, about 500 μm or less, about 400 μm or less, about 300 μm or less, about 250 μm or less, about 200 μm or less, about 150 μm or less, about 130 μm or less, about 100 μm or less, about 75 μm or less, about 60 μm or less, about 50 μm or less, about 40 μm or less, about 30 μm or less, about 25 μm or less, about 20 μm or less, or in ranges of about 20 μm to about 30 μm, about 25 μm to about 40 μm, about 30 μm to about 50 μm, about 40 μm to about 60 μm, about 50 μm to about 75 μm, about 60 μm to about 100 μm, about 75 μm to about 130 μm, about 100 μm to about 150 μm, about 130 μm to about 200 μm, about 150 μm to about 300 μm, about 200 μm to about 400 μm, about 300 μm to about 500 μm, about 400 μm to about 600 μm, about 500 μm to about 700 μm, about 600 μm to about 800 μm, about 700 μm to about 1 mm, about 800 μm to about 1.25 mm, about 1 mm to about 1.5 mm, or about 1.25 mm to about 2 mm. The relatively small thickness of the outer layer 111 decreases the overall volume of the outer porous material thereby decreasing the volume of bodily fluids that may be retained in the outer porous material. The decreasing volume of bodily fluids held within the outer porous material allows air flow through the chamber 104 to quickly evaporate the bodily fluids that are retained in the outer porous material thereby maintaining the porous material 110 dry. Further, decreasing the thickness of the outer layer 111 may allow the inner layer 114 to pull more bodily fluids from the outer porous material. It is noted that it has been found that increasing the thickness above about 1 mm may also adversely affect the flow of bodily fluids therethrough.

The outer porous material of the porous material 110 may be selected to exhibit a basis weight of about 10 gm/m² to about 20 g/m², about 15 gm/m² to about 25 g/m², about 20 g/m² to about 30 g/m², about 25 g/m² to about 35 g/m², about 30 g/m² to about 40 g/m², about 35 g/m² to about 45 g/m², about 40 g/m² to about 50 g/m², about 45 g/m² to about 55 g/m², about 50 g/m² to about 60 g/m², about 55 g/m² to about 70 g/m², about 60 g/m² to about 80 g/m², about 70 g/m² to about 90 g/m², about 80 g/m² to about 100 g/m², about 90 g/m² to about 110 g/m², about 100 g/m² to about 120 g/m², about 115 g/m² to about 125 g/m², about 120 g/m² to about 140 g/m², or about 130 g/m² to about 150 g/m². The basis weight of the outer porous material is a function of the density and thickness of the outer porous material. As such, the basis weight of the outer porous material may be selected for any of the same reasons as the density and thickness of the outer porous material.

As previously discussed, the outer porous material is formed from a plurality of fibers. The plurality of fibers may exhibit an average length and an average lateral dimension (e.g., diameter). In an example, the plurality of fibers may be selected to exhibit an average length that is about 500 μm to about 2 mm, about 1 mm to about 3 mm, about 2 mm to about 4 mm, about 3 mm to about 5 mm, about 4 mm to about 6 mm, about 5 mm to about 7 mm, about 6 mm to about 8 mm, about 7 mm to about 9 mm, about 8 mm to about 1 cm, about 9 mm to about 1.2 cm, about 1 cm to about 1.4 cm, about 1.2 cm to about 1.6 cm, about 1.4 cm to about 1.8 cm, about 1.6 cm to about 2 cm, about 1.8 cm to about 2.25 cm, about 2 cm to about 2.5 cm, about 2.25 cm to about 2.75 cm, about 2.5 cm to about 3 cm, about 2.75 cm to about 3.25 cm, about 3 cm to about 3.5 cm, about 3.25 cm to about 3.75 cm, about 3.5 cm to about 4 cm, about 3.75 cm to about 4.25 cm, about 4 cm to about 4.5 cm, about 4.25 cm to about 4.75 cm, about 4.5 cm to about 5 cm, about 4.75 cm to about 5.5 cm, about 5 cm to about 6 cm, about 5.5 cm to about 6.5 cm, about 6 cm to about 7 cm, about 6.5 cm to about 7.5 cm, about 7 cm to about 8 cm, about 7.5 cm to about 8.5 cm, about 8 cm to about 9 cm, about 8.5 cm to about 9.5 cm, or about 9 cm to about 10 cm. In an example, the fibers may exhibit an average lateral dimension that is about 1 μm to about 2 μm, about 1.5 μm to about 3 μm, about 2 μm to about 4 μm, about 3 μm to about 5 μm, about 4 μm to about 7 μm, about 6 μm to about 10 μm, about 8 μm to about 12.5 μm, about 10 μm to about 15 μm, about 12.5 μm to about 17.5 μm, about 15 μto about 20 μm, about 17.5 μm to about 25 μm, about 20 μm to about 30 μm, about 25 μm to about 35 μm, about 30 μm to about 40 μm, about 35 μm to about 45 μm, about 40 μm to about 50 μm, about 45 μm to about 55 μm, about 50 μm to about 60 μm, about 55 μm to about 65 μm, about 60 μm to about 70 μm, about 65 μm to about 75 μm, about 70 μm to about 80 μm, about 75 μm to about 85 μm, about 80 μm to about 90 μm, about 85 μm to about 95 μm, or about 90 μm to about 100 μm. The average length and average lateral dimension of the fibers may be selected such that the fibers exhibits an average aspect ratio. For example, the average length and average lateral dimension of the fibers may be selected such that the fibers exhibit an average aspect ratio (average length:average lateral dimension) of about 100:1 to about 200:1, about 150:1 to about 250:1, about 200:1 to about 300:1, about 250:1 to about 350:1, about 300:1 to about 400:1, about 350:1 to about 450:1, about 400:1 to about 500:1, about 450:1 to about 550:1, about 500:1 to about 600:1, about 550:1 to about 650:1, about 600:1 to about 700:1, about 650:1 to about 750:1, about 700:1 to about 800:1, about 750:1 to about 850:1, about 800:1 to about 900:1, about 850:1 to about 950:1, or about 900:1 to about 1,000:1.

The average length, average lateral dimension, and the average aspect ratio of the fibers may be selected based on a number of factors. In an example, increasing the aspect ratio (e.g., decreasing the average length and/or increasing the average lateral dimension) increases the durability of the outer porous material but may decrease the strength of the outer porous material. In an example, increasing the aspect ratio (e.g., increasing average length) of the fibers may increase the mechanical binding of the fibers. For instance, increasing the aspect ratio of the fibers facilitates entanglement of the fibers which increases the strength and durability of the outer porous material. The entanglement of the fibers may also preclude or minimize the amount of other binding techniques that are applied to the outer porous material, such as heat, chemical binding, or other mechanical binding (e.g., further entanglement caused by needle punching or high pressure water jets). However, increasing the aspect ratio of the fibers may make dispersion of the fibers more difficult (e.g., uniformity of the outer porous material difficult). Further, increasing the aspect ratio may limit the type of nonwoven webs that may form the outer porous material. For instance, fibers with large average lengths (e.g., large aspect ratios) may not be used in carded webs and may have to be used in air laid webs. In an example, decreasing the aspect ratio may decrease the entanglement of the fibers thereby necessitating further binding of the fibers. As such, the average length, average lateral dimension, and average aspect ratio of the fibers may be selected based on the desired strength, mechanical binding between the fibers, the amount of processing of the outer porous material (e.g., is further processing to increasing the binding via heat, etc. desired), the type of nonwoven web that includes the fibers, the uniformity of the fibers, etc.

Generally, the average person discharges urine at a rate of about 6 ml/s to about 50 ml/s, such as at a rate of about 10 ml/s to about 25 ml/s. The rate at which the person urinate may vary, such as based on the size of the person and the age of the person. The outer porous material may be selected to receive bodily fluids and have the bodily fluids flow through a portion thereof at a rate that is comparable to the rate at which the individual discharged bodily fluids to prevent leaks. For example, the outer porous material may be selected to receive bodily fluids and have the bodily fluids flow through a portion thereof at a rate that is greater than about 6 ml/s, greater than about 10 ml/s, greater than about 20 ml/s, greater than about 30 ml/s, greater than about 40 ml/s, greater than about 50 ml/s, or in ranges of about 6 ml/s to about 10 ml/s, about 8 ml/s to about 12 ml/s, about 10 ml/s to about 15 ml/s, about 12.5 ml/s to about 17.5 ml/s, about 15 ml/s to about 20 ml/s, about 17.5 ml/s to about 22.5 ml/s, about 20 ml/s to about 25 ml/s, about 22.5 ml/s to about 27.5 ml/s, about 25 ml/s to about 30 ml/s, about 27.5 ml/s to about 35 ml/s, about 30 ml/s to about 40 ml/s, about 35 ml/s to about 45 ml/s, or about 40 ml/s to about 50 ml/s.

The rate at which the outer porous material receives bodily fluids and has the bodily fluids flow through a portion thereof may depend on a number of factors. In an example, the rate at which the outer porous material receives bodily fluids and has the bodily fluids flow through a portion thereof may depend inversely on the density and weight basis of the outer porous material, wherein increasing the density and/or weight basis of the outer porous material may decrease the rate at which the outer porous material receives bodily fluids and has the bodily fluids flow through a portion thereof and vice versa. In an example, the rate at which the outer porous material receives bodily fluids and has the bodily fluids flow through a portion thereof may depend on the hydrophilicity of the outer porous material. In an example, the rate at which the outer porous material receives bodily fluids and has the bodily fluids flow through a portion thereof may depend on the type of nonwoven web (e.g., carded web, needle punched web, etc.) since each type of nonwoven web may exhibit different rate at which the outer porous material captures and transports the bodily fluids.

As previously discussed, the outer porous material may be formed from at least one nonwoven web. The outer porous material may be formed from any suitable nonwoven web. In an embodiment, the nonwoven web of the outer porous material includes at least one carded web. The carded web includes a plurality of fibers that may be generally oriented in the same direction. The generally same orientation of the fibers of the carded web cause the carded web to be anisotropic. For example, the strength of the carded web is greatest when a force applied thereto is generally parallel to the fibers but the strength of the carded web decreases as the force applied thereto becomes more oblique or perpendicular to the orientation of the fibers. As such, the carded web may need to be positioned in the chamber 104 to mitigate forces being applied to the carded web that are not generally parallel to the orientation of the fibers or requires addition binding between the fibers (e.g., heat or chemical) to prevent unsatisfactory wear of the carded web. The initial flow the bodily fluids through the carded web may vary depending on whether the bodily fluids are flowing parallel, obliquely, or perpendicular to the orientation of the fibers. As such, selecting the nonwoven web to include the carded web allows for selecting the strength and flow characteristics of the porous material 110 based on the orientation of the fibers. Even though the fibers are generally oriented, the orientation of each of the fibers may slightly vary which causes the porosity of the carded web to be sufficiently high that the carded web exhibits any of the density, thickness, basis weight, and flow rates disclosed herein.

In an embodiment, the nonwoven web of the outer porous material may include at least one needle punched web. The needle punched web may be formed from a sheet including a plurality of fibers. The sheet may include a plurality of randomly oriented fibers (e.g., the fibers are generally parallel to and randomly oriented in the plane), or generally oriented fibers (e.g., a carded web) since the orientation of the fibers may better facilitate flow of the bodily fluids therethrough. A plurality of needles (e.g., a plurality of barbed needles) are inserted into the sheet in a direction that is generally parallel to a thickness of the sheet which causes some of the fibers to become entangled and interlocked. For example, insertion of the needles into the sheet cause some of the fibers to reorient and migrate from the surface of the sheet to an interior thereof to form columns. The entanglement of the fibers caused by the insertion of the needles may sufficiently entangle the fibers such that no additional binding is necessary to bond the fibers together. The entanglement of the fibers may cause the needle punched web to exhibit more isotropic properties compared to the carded web and, thus, may not require specific orientation in the chamber 104 or additional binding of the fibers. The needle punched web may exhibit good flow features. For example, the needles extending into the sheet may form divots which facilitate flow of the bodily fluids vertically through the needle punched web.

In an embodiment, the nonwoven web of the outer porous material may include at least one air laid web. The air laid web may exhibit a plurality of randomly oriented fibers. The plurality of random fibers may exhibit a length that is sufficiently large that the fibers become entangled and do not need be bounded together or the fibers may be bonded. Due to the random orientation of the fibers, the air laid web tends to be isotropic and exhibit a high porosity. Similar, due to the random orientation of the fibers, the air laid web may exhibit a high loft. The air laid web may be formed from fibers that cannot be carded (e.g., short fibers).

In an embodiment, the nonwoven web of the outer porous material may include at least one spunbonded web. The spunbonded web is formed by tangling the fibers before depositing the fibers on a belt. The belt then carries the fibers to a device that bonds (e.g., thermally, mechanically, or chemically bonds) the fibers together. Generally, the fibers are randomly oriented in the spunbonded web though the fibers may be slightly biased in the direction that the belt moves. Due to the random orientation of the fibers, the air laid web tends to be isotropic and exhibit a high porosity. Similar, due to the random orientation of the fibers, the air laid web may exhibit a high loft. The spunbonded web may exhibit relatively good water absorption. The spunbonded web may exhibit a durability that is less than at least some of the nonwoven webs disclosed herein. In a particular example, the nonwoven web may be a spunbonded web when the outer porous material includes bamboo since a spunbonded web including bamboo exhibits better fluid permeability, better wicking, and retains less bodily fluids than if the spunbonded web including at least some other materials or the bamboo was included in at least some other nonwoven web.

In an embodiment, the nonwoven web of the outer porous material may include at least one spunlaced web. The spunlaced web is formed by providing a sheet that includes randomly oriented fibers or a carded web. High pressure water jets that are generally parallel to the thickness of the sheet are directed towards the sheet. Similar to the needle punched web, the high pressure jets of water cause some of the fibers to migrate from an exterior of the sheet to an interior thereof to form columns. Thus, the spunlaced web may function similar to the needle punched web, namely that the spunlaced web may be more isotropic than the carded web and includes divots. However, the spunlaced web may exhibit at least one of a density that is less than, a thickness that is greater than, or a base weight that is less than the needle punched web. As such, the spunlaced web may be more delicate (e.g., less durable or softer) than the needle punched web. The more delicate spunlaced web may more comfortably contact the skin of the patient than the needle punched web.

In an embodiment, the nonwoven web of the outer porous material may include at least one vertical lapped nonwoven fabric. The vertical lapped nonwoven fabric is formed by lapping a sheet vertically such that a cross-section of the vertical lapped nonwoven fabric taken along a plane that is parallel to the thickness and length of the vertical lapped nonwoven fabric shows a periodic wavy (e.g., sinusoidal) structure. The folds in the sheet cause the fibers of the nonwoven web to be preferentially oriented vertically between the folds and the fibers at the folds to be preferentially oriented horizontally. Thus, the vertically lapped nonwoven fabric causes the bodily fluids to wick both horizontally and vertically. The vertically oriented fibers also cause the vertical lapped nonwoven fabric to be resistant to collapse, even at high vacuum pressures. Similarly, the vertically oriented fibers also cause the vertical lapped nonwoven fabric to exhibit excellent elastic recovery and localized deformation when a force is applied thereto that is generally parallel to the vertically oriented fibers. The elastic recovery and localized deformation minimize the likelihood that the nonwoven material collapses when both a suction force and an external force is applied to the nonwoven web (e.g., laying on or pressing against fluid collection assembly 100) and increases the likelihood that any collapse is localized and temporary. The vertical lapped nonwoven fabric may also exhibit low density and is highly moldable. The vertical lapped nonwoven fabric may exhibit any suitable thickness by increasing or decreasing the distance between folds.

In an embodiment, the nonwoven web of the outer porous material may include at least one horizontal lapped nonwoven fabric. The horizontal lapped nonwoven fabric is formed by lapping a sheet horizontally. The folds in the sheet cause the fibers of the nonwoven web to be preferentially oriented horizontally between the folds and the fibers at the folds to be preferentially oriented vertically. Thus, the horizontal lapped nonwoven fabric causes the bodily fluids to wick both horizontally and vertically. The horizontal lapped nonwoven fabric may exhibit high thickness by merely increasing the number of folds formed therein.

In an embodiment, the nonwoven web of the outer porous material may include at least one crossed lapped nonwoven fabric. The crossed lapped nonwoven fabric is substantially similar to the horizontal lapped nonwoven fabric except that each layer is not parallel to the adjacent layers. Instead, each layer extends obliquely relative to the previously layer which causes the crossed lapped nonwoven fabric to exhibit more isotropic properties than the horizontal lapped nonwoven web, especially when the horizontal and crossed lapped nonwoven fabrics are formed from a carded web.

It is currently believed that the carded web, needle punched web, the air laid web, the spunlaced web, spunbonded web, vertical lapped nonwoven web, horizontal lapped nonwoven web, and the cross lapped nonwoven web, are the preferred nonwoven webs to be included in the outer porous material. However, it is noted that the outer porous material may include one or more nonwoven webs other than the carded web, needle punched web, the air laid web, and the spunlaced web. In an example, the outer porous material may include a wet laid web even though the wet laid web may exhibit low durability compared to the other nonwoven webs disclosed herein. In an example, the outer porous material may include meltblown nonwoven webs even though such nonwoven webs may exhibit too low of porosity for some applications.

In an embodiment, the outer porous material may include a woven fabric instead of or in addition to a nonwoven web. Forming the outer porous material from a woven material may increase the durability of the porous material 110 than if the outer porous material is formed from a nonwoven material. However, forming the outer porous material from a woven material may decrease the compressibility of the porous material 110 thereby making the porous material 110 less comfortable and may make conforming the porous material 110 to the vaginal region, which limits leaks, more difficult. Also, it is more difficult to from a woven outer porous material than a nonwoven outer porous material. As such, using the woven outer porous material may cause logistic issues, increase manufacturing difficulties, and increase cost.

Selecting the outer layer 111 to include the outer porous material facilitates flow of the bodily fluids therethrough. In other words, the outer porous material facilitates relatively quick flow of the bodily fluids from an outer surface of the outer layer 111 proximate to the urethral opening to an inner surface of the outer layer 111 that abuts the inner layer 112. The relatively quick flow of the bodily fluids through the outer porous material allows the bodily fluids to quickly flow to the inner layer 112. It has been surprising found that the outer porous material allows the bodily fluids to quickly flow into the inner layer 112 from the outer porous material, even when the inner layer 112 includes a hydrophobic material.

The outer layer 111 may be disposed on an outer surface of the inner layer 112. In an embodiment, the outer layer 111 is positioned on the inner layer 112 to prevent or at least minimize formation of air gaps between the outer layer 111 and the inner layer 112. As used herein, air gaps refers to unoccupied gaps between the outer layer 111 and the inner layer 112 that are significantly larger (e.g., at least 5 times larger or at least 10 times larger) than the combined average pore size of the outer layer 111 and the inner layer 112. In an example, the outer layer 111 is disposed on the inner layer 112 such that at least most 10% (e.g., at most 7.5%, at most 5%, at more 3%, at more 2%, or at most 1%) of the surface area of the inner layer 112 adjacent to the outer layer 111 has an air gap adjacent thereto. As previously discussed, it has been found that the bodily fluids received by the outer porous material of the outer layer 111 flow relatively freely from the outer porous material into an adjacent porous material. However, the air gaps between the outer layer 111 and the inner layer 112 form barriers that inhibit flow of the bodily fluids from the outer layer 111 and the inner layer 112. As such, preventing or at least minimizing the formation of air gaps between the outer layer 111 and the inner layer 112 improves flow of the bodily fluids between the outer layer 111 and the inner layer 112. In an example, the outer layer 111 may be tightly wrapped around the inner layer 112 to prevent the formation of air gaps therebetween.

The inner layer 112 is distinct from the outer layer 111. The inner layer 112 is configured to support the outer layer 111 since the outer layer 111 may be formed from a relatively foldable, flimsy, or otherwise easily deformable material. For example, the inner layer 112 may be positioned such that the outer layer 111 is disposed between the inner layer 112 and the fluid impermeable barrier 102. As such, the inner layer 112 may support and maintain the position of the outer layer 111. The inner layer 112 may include any material that may wick, absorb, adsorb, or otherwise allow fluid transport of the bodily fluids, such as any of the fluid outer porous materials disclosed herein above. For example, the outer porous material(s) may be utilized in a more dense or rigid form than in the outer layer 111 when used as the inner layer 112. The inner layer 112 may be formed from any fluid permeable material that is less deformable than the outer layer 111. For example, the inner layer 112 may include a porous polymer (e.g., nylon, polyester, polyurethane, polyethylene, polypropylene, polyvinyl chloride, etc.) structure or an open cell foam. In an example, the inner layer 112 may include spun nylon fiber, a polyurethane foam, a polyethylene foam, or a polyvinyl chloride foam. In some examples, the inner layer 112 may include a nonwoven (e.g., a vertical nonwoven web or any other nonwoven web disclosed herein) or woven material. In some examples, the inner layer 112 may be formed from a natural material, such as cotton, wool, silk, bamboo, or combinations thereof. In such examples, the material may have a coating to prevent or limit absorption of fluid into the material, such as a water repellent coating. In some examples, the inner layer 112 may be formed from fabric, felt, gauze, or combinations thereof.

In an embodiment, the inner layer 112 may be configured to wick any bodily fluids away from the outer layer 111, thereby preventing the bodily fluids from escaping the chamber 104. The permeable properties referred to herein may be wicking, capillary action, diffusion, or other similar properties or processes, and are referred to herein as “permeable” and/or “wicking.” Such “wicking” and/or “permeable” properties may not include absorption of the bodily fluids into at least a portion of the inner layer 112, such as not include adsorption of the bodily fluids into the inner layer 112. Put another way, substantially no absorption or solubility of the bodily fluids into the material may take place after the material is exposed to the bodily fluids and removed from the bodily fluids for a time. While no absorption or solubility is desired, the term “substantially no absorption” may allow for nominal amounts of absorption and/or solubility of the bodily fluids into the inner layer 112 (e.g., absorbency), such as less than about 30 wt % of the dry weight of the inner layer 112, less than about 20 wt %, less than about 15 wt %, less than about 10 wt %, less than about 7 wt %, less than about 5 wt %, less than about 3 wt %, less than about 2 wt %, less than about 1 wt %, or less than about 0.5 wt % of the dry weight of the inner layer 112. The inner layer 112 may also wick the bodily fluids generally towards an interior of the chamber 104, as discussed in more detail below. In an embodiment, the inner layer 112 may include at least one absorbent or adsorbent material.

In an embodiment, at least a portion of the inner layer 112 may be hydrophobic. The inner layer 112 may be hydrophobic when the inner layer 112 exhibits a contact angle with water (a major constituent of bodily fluids) that is greater than about 90°, such as in ranges of about 90° to about 120°, about 105° to about 135°, about 120° to about 150°, about 135° to about 175°, or about 150° to about 180°. The hydrophobicity of the inner layer 112 may limit absorption, adsorption, and solubility of the bodily fluids in the inner layer 112 thereby decreasing the amount of bodily fluids held in the inner layer 112. The lower hydrophobicity of the ionner layer 111 may help the porous material 110 receive the bodily fluids from the urethral opening while the hydrophobicity of the inner layer 112 limits the bodily fluids that are retained in the porous material 110.

The inner layer 112 may exhibit a thickness (e.g., radius and/or diameter) that is about 1 mm or greater, about 2 mm or greater, about 4 mm or greater, about 6 mm or greater, about 8 mm or greater, about 10 mm or greater, about 12 mm or greater, about 14 mm or greater, about 16 mm or greater, about 18 mm or greater, about 20 mm or greater, about 22 mm or greater, about 25 mm or greater, or in ranges of about 1 mm to about 4 mm, about 2 mm to about 6 mm about 4 mm to about 8 mm, about 6 mm to about 10 mm, about 8 mm to about 12 mm, about 10 mm to about 14 mm, about 12 mm to about 16 mm, about 14 mm to about 18 mm, about 16 mm to about 20 mm, about 18 mm to about 22 mm, or about 20 mm to about 25 mm. Generally, increasing the thickness of the inner layer 112 increases the quantity of bodily fluids that may be temporarily stored therein and may flow therethrough thereby decreasing the likelihood that the fluid collection assembly 100 leaks. However, increasing the thickness of the inner layer 112 may dilute any suction force applied to the chamber 104 and may make it difficult to position the fluid collection assembly 100 adjacent to a urethral opening.

In an embodiment, the inner layer 112 includes at least one inner porous material. As used herein, the inner porous material includes at least one of a vertical lapped nonwoven material, a polyurethane foam, a polyvinyl chloride foam, or a polyethylene foam. The inner porous material is able to quickly receive bodily fluids from the individual, even when the individual discharges a large quantity of bodily fluids over a short period of time. In an example, the inner porous material may facilitate moving the bodily fluids through the chamber of the fluid collection assembly and towards an outlet (e.g., the fluid outlet or an inlet of a conduit disposed through the fluid outlet) which allows the porous material 110 to remain dry. Further, it has been surprisingly found that the bodily fluids received into the outer porous material may flow easily from the outer porous material into the inner porous material and the inner porous material pulls bodily fluids from the outer porous material that would otherwise remain in the polyurethane foam.

When the inner porous material include a foam (i.e., at least one of the polyurethane foam, the polyethylene foam, or the polyvinyl chloride foam), the inner porous material may exhibit about 5 pores/cm² to about 20 pores/cm², such as about 5 pores/cm² to about 7 pores/cm², about 6 pores/cm²to about 8 pores/cm², about 7 pores/cm² to about 9 pores/cm², about 8 pores/cm²to about 10 pores/cm², about 9 pores/cm² to about 11 pores/cm², about 10 pores/cm²to about 12 pores/cm², about 11 pores/cm² to about 13 pores/cm², about 12 pores/cm²to about 14 pores/cm², about 13 pores/cm² to about 15 pores/cm², about 14 pores/cm²to about 16 pores/cm², about 15 pores/cm² to about 17 pores/cm², about 16 pores/cm²to about 18 pores/cm², about 17 pores/cm² to about 19 pores/cm², or about 18 pores/cm²to about 20 pores/cm². Generally, increasing the number of pores/cm² of the foam increases the number of interconnected pores formed in the porous material, increases the quantity of bodily fluids that may be stored in the foam, and the quantity of and rate at which the bodily fluids may flow through the foam. However, increasing the number of pores/cm² decreases the strength of the foam. As such, the number of pores/cm² of the foam may be selected based on balancing these factors.

When the inner porous material includes a foam (i.e., at least one of the polyurethane foam, the polyethylene foam, or the polyvinyl chloride foam), the inner porous material may exhibit a density of about 75 kg/m³ to about 200 kg/m³, such as about 75 kg/m³ to about 90 kg/m³, about 80 kg/m³ to about 100 kg/m³, about 90 kg/m³ to about 110 kg/m³, about 100 kg/m³ to about 120 kg/m³, about 110 kg/m³ to about 130 kg/m³, about 120 kg/m³ to about 140 kg/m³, about 130 kg/m³ to about 150 kg/m³, about 140 kg/m³ to about 160 kg/m³, about 150 kg/m³ to about 170 kg/m³, about 160 kg/m³ to about 180 kg/m³, about 170 kg/m³ to about 190 kg/m³, or about 180 kg/m³ to about 200 kg/m³. Generally, increasing the density of the inner porous material increases the strength of the inner porous material. However, increasing the density of the inner porous material may decrease the porosity of the inner porous material which decreases the quantity of bodily fluids that may be temporarily stored in the porous material 110 and decrease the flow rate of the bodily fluids through the inner porous material. As such, the density of the inner porous material may be selected based on balancing the desired strength, porosity, and flow rate of the bodily fluids through the inner porous material.

When the inner porous material includes a vertical lapped nonwoven material, the inner porous material may exhibit a density of about 50 kg/m²·cm or greater, about 75 kg/m²·cm or greater, about 100 kg/m²·cm or greater, about 125 kg/m²·cm or greater, about 150 kg/m²·cm or greater, about 175 kg/m²·cm or greater, about 200 kg/m²·cm or greater, about 250 kg/m²·cm or greater, about 300 kg/m²·cm or greater, or in ranges of about 50 kg/m²·cm to about 100 kg/m²·cm, about 75 kg/m²·cm to about 125 kg/m²·cm, about 100 kg/m²·cm to about 150 kg/m²·cm, about 125 kg/m²·cm to about 175 kg/m²·cm, about 150 kg/m²·cm to about 200 kg/m²·cm, about 175 kg/m²·cm to about 250 kg/m²·cm, or about 200 kg/m²·cm to about 300 kg/m²·cm. Generally, increasing the density of the inner porous material increases the strength of the inner porous material. However, increasing the density of the inner porous material may decrease the porosity of the inner porous material which decreases the quantity of bodily fluids that may be temporarily stored in the porous material 110 and decrease the flow rate of the bodily fluids through the inner porous material. As such, the density of the inner porous material may be selected based on balancing the desired strength, porosity, and flow rate of the bodily fluids through the inner porous material.

In some embodiments, one of the outer layer 111 or the inner layer 112 may be omitted from the porous material 110. In some embodiments, the porous material 110 may include one or more additional layers instead of or in addition to at least one of the outer layer 111 or the inner layer 112.

The porous material 110 may at least substantially completely fill the portions of the chamber 104 that are not occupied by the conduit 114. In some examples, the porous material 110 may not substantially completely fill the portions of the chamber 104 that are not occupied by the conduit 114. In such an example, the fluid collection assembly 100 includes the reservoir 120 disposed in the chamber 104.

The reservoir 120 is a substantially unoccupied portion of the chamber 104. The reservoir 120 may be defined between the fluid impermeable barrier 102 and porous material 110. The bodily fluids that are in the chamber 104 may flow through the porous material 110 to the reservoir 120. The reservoir 120 may retain of the bodily fluids therein.

The bodily fluids that are in the chamber 104 may flow through the porous material 110 to the reservoir 120. The fluid impermeable barrier 102 may retain the bodily fluids in the reservoir 120. While depicted in the distal end region 122, the reservoir 120 may be located in any portion of the chamber 104 such as the proximal end region 124. The reservoir 120 may be located in a portion of the chamber 104 that is designed to be located in a gravimetrically low point of the fluid collection assembly when the fluid collection assembly is worn.

In some examples (not shown), the fluid collection assembly 100 may include multiple reservoirs, such as a first reservoir that is located at the portion of the chamber 104 closest to the inlet of the conduit 114 (e.g., distal end region 122) and a second reservoir that is located at the portion of the of the chamber 104 that is at or near proximal end region 124). In another example, the porous material 110 is spaced from at least a portion of the conduit 114, and the reservoir 120 may be the space between the porous material 110 and the conduit 114.

The conduit 114 may be at least partially disposed in the chamber 104. The conduit 114 may be used to remove the bodily fluids from the chamber 104. The conduit 114 includes at least one wall defining an inlet 116, an outlet (not shown) downstream from the inlet 116, and a passageway. The outlet of the conduit 114 may be operably coupled to a vacuum source, such as a vacuum pump for withdrawing fluid from the chamber 104 through the conduit 114. For example, the conduit 114 may extend into the fluid impermeable barrier 102 from the proximal end region 124 and may extend to the distal end region 122 to a point proximate to the reservoir 120 therein such that the inlet 116 is in fluid communication with the reservoir 120. The conduit 114 fluidly couples the chamber 104 with the fluid storage container (not shown) or the vacuum source (not shown).

The conduit 114 may extend through a bore in the porous material 110. In an embodiment, the conduit 114 extends from the fluid outlet 108, through the bore, to a location that is proximate to the reservoir 120. In such an embodiment, the inlet 116 may not extend into the reservoir 120 and, instead, the inlet 116 may be disposed within the porous material 110 or at a terminal end thereof. For example, an end of the conduit 114 may be coextensive with or recessed within the porous material 110. In an embodiment, the conduit 114 is at least partially disposed in the reservoir 120 and the inlet 116 may be extended into or be positioned in the reservoir 120. The bodily fluids collected in the fluid collection assembly 100 may be removed from the chamber 104 via the conduit 114.

Locating the inlet 116 at or near a location expected to be the gravimetrically low point of the chamber 104 when worn by an individual enables the conduit 114 to receive more of the bodily fluids than if inlet 116 was located elsewhere and reduce the likelihood of pooling (e.g., pooling of the bodily fluids may cause microbe growth and foul odors). For instance, the bodily fluids in the porous material 110 may flow in any direction due to capillary forces. However, the bodily fluids may exhibit a preference to flow in the direction of gravity, especially when at least a portion of the porous material 110 is saturated with the bodily fluids. Accordingly, one or more of the inlet 116 or the reservoir 120 may be located in the fluid collection assembly 100 in a position expected to be the gravimetrically low point in the fluid collection assembly 100 when worn by an individual, such as the distal end region 122.

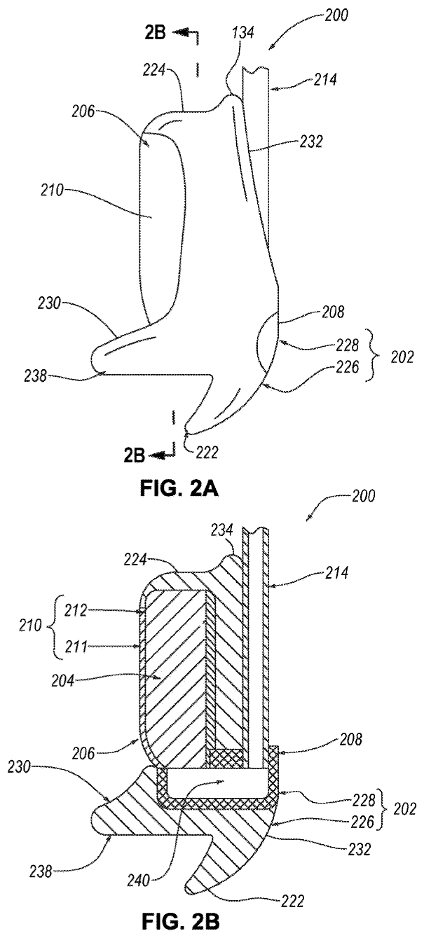

The inlet 116 and the outlet of the conduit 114 are configured to fluidly couple (e.g., directly or indirectly) the vacuum source (not shown) to the chamber 104 (e.g., the reservoir 120). As the vacuum source (FIG. 3) applies a suction force in the conduit 114, the bodily fluids in the chamber 104 (e.g., at the distal end region 122 such as in the reservoir 120) may be drawn into the inlet 116 and out of the fluid collection assembly 100 via the conduit 114. In some examples, the conduit 114 may be frosted or opaque (e.g., black) to obscure visibility of the bodily fluids therein.

As previously discussed, the conduit 114 may be configured to be at least insertable into the chamber 104. In an example, the conduit 114 may be positioned in the chamber 104 such that a terminal end of the conduit 114 is spaced from the fluid impermeable barrier 102 or other components of the fluid collection assembly 100 that may at least partially obstruct or block the inlet 116. Further, the inlet 116 of the conduit 114 may be offset relative to a terminal end of the porous material 110 such that the inlet 116 is closer to the proximal end region 124 of the fluid collection assembly 2100 than the terminal end of the porous material 110. Offsetting the inlet 116 in such a manner relative to the terminal end of the porous material 110 allows the inlet 116 to receive bodily fluids directly from the porous material 110 and, due to hydrogen bonding, pulls more bodily fluids from the porous material 110 into the conduit 114.

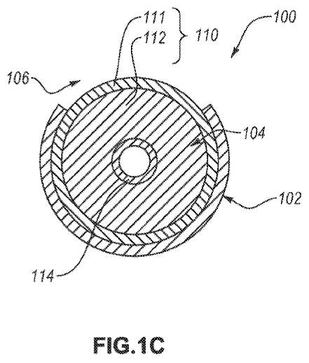

FIG. 2A is an isometric view of a fluid collection assembly 200, according to an embodiment. FIG. 2B is a cross-sectional schematic of the fluid collection assembly 200 taken along plane 2B-2B shown in FIG. 2A. The fluid collection assembly 200 is example of a female fluid collection assembly for receiving and collecting bodily fluids from a female. Except as otherwise disclosed herein, the fluid collection assembly is the same as or substantially similar to any of the fluid collection assemblies disclosed herein. The fluid collection assembly 200 includes a fluid impermeable barrier 202 that is the same or similar to any of the fluid impermeable barriers disclosed herein in one or more aspects. The fluid impermeable barrier 302 at least defines a chamber 204, at least one opening 206, and a fluid outlet 208. The fluid collection assembly 200 also includes at least one porous material 210 positioned within the chamber 204. The porous material 210 may be the same or similar to any of the porous materials disclosed herein in one or more aspects (e.g., the porous material 210 includes an outer layer 211 include an outer porous material and an inner layer 212). The fluid collection assembly 200 may also include at least one conduit 214 partially positioned within the fluid outlet 208 that is configured to remove one or more bodily fluids from the chamber 204. The conduit 214 may not extend through the porous material 210.

In an embodiment, the fluid impermeable barrier 202 may include a shell 226 and a connector piece 228 secured to the shell 226. The shell 226 of the fluid collection assembly 200 includes a proximal end region 224, a distal end region 222 opposite the proximal end region 224, a front side 230, and a back side 232 opposite the front side 230. Generally, during use, the distal end region 222 is closer to the gluteal cleft of the individual than the proximal end region 224 and the front side 230 generally faces the vaginal region of the individual. The shell 226 may be formed from silicone, neoprene, a thermoplastic elastomer, or other fluid impermeable material.

In an embodiment, the shell 226 includes one or more flanges. The flanges may provide more locations for underwear or other clothing to contact and press against the fluid collection assembly 200 which may facilitate securing the fluid collection assembly 200 to the vaginal region of the individual and may improve patient comfort. In an embodiment, the flanges may include at least one of an upper flange 234 forming the proximal end region 224 and a bottom flange 236 opposite the upper flange 234 that forms the distal end region 222.

The flanges of the body may extend from the rest of the shell 226 by a distance that is about 1 mm or greater, about 1 mm or greater, about 3 mm or greater, about 4 mm or greater, about 5 mm or greater, about 6 mm or greater, about 7.5 mm or greater, about 1 cm or greater, about 1.25 cm or greater, about 1.5 cm or greater, about 1 cm or greater, about 1.5 cm or greater, about 3 cm or greater, about 4 cm or greater, about 5 cm or greater, or in ranges of about 1 mm to about 3 mm, about 1 mm to about 4 mm, about 3 mm to about 5 mm, about 4 mm to about 6 mm, about 5 mm to about 7.5 mm, about 6 mm to about 1 cm, about 7.5 mm to about 1.25 cm, about 1 cm to about 1.5 cm, about 1.25 cm to about 1 cm, about 1.5 cm to about 1.5 cm, about 1 cm to about 3 cm, about 1.5 cm to about 4 cm, or about 3 cm to about 5. The distance that the flanges extend from the rest of the shell 226 may be selected based on the expected size of the vaginal region of the individual (e.g., larger flanges for a larger vaginal region) or the expect rotational forces applied to the fluid collection assembly 200 during use. In some 10 examples, at least some of the flanges may extend further from the rest of the shell 226 that other flanges. For instance, as illustrated, the bottom flange 236 may extend further from the rest of the fluid impermeable barrier 202 than the upper flange 234 since some individuals may find the longer bottom flange 236 more comfortable.

In an embodiment, the one or more flanges may exhibit a concave curve relative to the front side 230 of the shell 226. The concave curve of the flanges may extend from the proximal end region 224 to the distal end region 222. The concave curve of the flanges may allow the flanges to better conform to the shape of the vaginal region since the vaginal region is curved. Conforming the flanges to the shape of the vaginal region may make the fluid collection assembly 200 more comfortable by more uniformly 20 distributing pressure across the vaginal region, especially when the flanges contact the labia majora

In an embodiment, the shell 226 may include a sump 238 at or near the distal end region 222. The sump 238 may extend outwardly from the front side 230 of the shell 226. During use, the sump 238 is configured to be at, near, or otherwise in fluid communication with a gravimetric low point of the porous material 210. For example, the sump 238 may receive a portion of the porous material 210 therein. The sump 238 may receive at least some of the bodily fluids that are received by the porous material 210. The sump 238 may prevent or at least inhibit bodily fluids from leaking from the fluid collection assembly 200. The sump 238 may include at least a portion of the connector piece 228 at least partially disposed therein.

In an embodiment, the shell 226 may define a recess that is configured to receive a conduit 214. The recess may extend from or near the proximal end region 224 to or near the distal end region 222 thereby allowing the conduit 214 to extend from or near the individual's abdominal region to the connector piece 228. In an embodiment, the recess may be configured such that the shell 226 encloses and/or abuts less than 50% of a circumference of the conduit 214, thereby allowing the conduit 214 to freely enter and leave the recess during use. Allowing the conduit 214 to freely enter and leave the recess may facilitate positioning of the fluid collection assembly 200 such that the porous material 210 is adjacent to the vaginal region even when the conduit 214 is bending away from the vaginal region. Also, allowing the conduit 214 to freely enter and leave the recess may increase the likelihood that movement of the conduit 214 does not move the porous material 210 relative to the vaginal region since movement of the porous material 210 may cause leaking. In an embodiment, at least a portion of the recess may be configured such that the shell 226 encloses and/or abuts more than 50% (e.g., 51% to about 55%, about 53% to about 57%, or about 55% to about 60%) of the circumference of the conduit 214. Enclosing more than 50% of the circumference of the conduit 214 may more securely attach the conduit 214 to the shell 226 and may allow the conduit 214 to provide additional structure to the shell 226. The percentage of the conduit 214 enclosed and/or abutted by the shell 226 may be selected such that the inherent elasticity of the shell 226 and the conduit 214 allows the conduit 214 to be easily snapped into and out of the recess. As such, the conduit 214 may be removed from the recess to facilitate positioning the porous material 210 adjacent to the vaginal region or when the conduit 214 is moved.

As previously discussed, the fluid impermeable barrier 202 includes a connector piece 228 that is attached to (e.g., with an adhesive, welding, interference fit, etc.) or integrally formed with the shell 226. The connector piece 228 is positioned at or near the distal end region 222 of the shell 226 which allows the connector piece 228 to receive bodily fluids that flow to the gravimetric low point of the porous material 210. In an embodiment, a portion of the connector piece 228 may be positioned in the sump 238 of the shell 226. In an embodiment, not shown, the connector piece 228 may form the sump 238 instead of the shell 226.

The connector piece 228 is configured to be connected to the conduit 214. As such, the connector piece 228 may define the fluid outlet 208 which configured to be attached to or otherwise in fluid communication with the conduit 214. The fluid outlet 208 may be positioned adjacent to the back side 232 of the fluid impermeable barrier 222. The connector piece 228 may also define a channel 240 (e.g., tube) configured to allow the porous material 310 and the sump 238 to be in fluid communication with the conduit 214.

In an embodiment, at least a portion of the connector piece 228 may exhibit a rigidity that is greater than the shell 226. The increased rigidity of the connector piece 228 relative to the shell 226 may facilitate attachment of the conduit 214 to the connector piece 228. In an example, the connector piece 228 may exhibit a rigidity that is greater than the shell 226 when the connector piece 228 is formed from a material exhibiting at least one of a greater Young's modulus (i.e., modulus of elasticity), yield strength, or ultimate tensile strength than a material that forms the shell 226. In an example, the connector piece 228 may exhibit a rigidity that is greater than the shell 226 when the connector piece 228 exhibits a thickness that is greater than the shell 226.

The fluid collection assembly shown in FIGS. 1A-2B are examples of female fluid collection assemblies that are configured to collect bodily fluids from females (e.g., collect urine from a female urethra). However, the fluid collection assemblies, systems, and methods disclosed herein may include male fluid collection assemblies shaped, sized, and otherwise configured to collect bodily fluids from males (e.g., collect urine from a male urethra). FIG. 3 is a cross-sectional schematic of male fluid collection assembly 300, according to an embodiment.

The fluid collection assembly 300 includes a base 342 (e.g., annular base) and a sheath 344. The base 342 is sized, shaped, and made of a material to be coupled to skin that surrounds the male urethral opening (e.g., penis) and have the male urethral opening positioned therethrough. For example, the base 342 may define an aperture 346. The base 342 is sized and shaped to be positioned around the male urethral opening (e.g., positioned around and/or over the penis) and the aperture 346 may be configured to have the male urethral opening positioned therethrough. The base 342 may also be sized, shaped, made of a material, or otherwise configured to be coupled (e.g., adhesively attached, such as with a hydrogel adhesive) to the skin around the male urethral opening (e.g., around the penis). In an example, the base 342 may exhibit the general shape or contours of the skin surface that the base 342 is selected to be coupled with. The base 342 may be flexible thereby allowing the base 342 to conform to any shape of the skin surface. The base 342 may include a laterally (e.g., radially) extending flange 347. The base 342 also defines a hollowed region that is configured to receive (e.g., seal against) the sheath 344. For example, the base 342 may include a longitudinally extending flange 348 that extends upwardly from the base 342. The longitudinally extending flange 348 may be tall enough to prevent the sheath 344 from being accidentally removed from the base 342 (e.g., at least 0.25 cm tall, 1 cm tall, at least 3 cm tall, or at least 5 cm tall). The base 342 is located at a proximal end region 324 (with respect to a wearer) of the fluid collection assembly 300.

The sheath 344 includes (e.g., may be formed from) a fluid impermeable barrier 302 that is sized and shaped to fit into the hollowed region of the base 342. For example, the sheath 344 may be generally tubular or cup-shaped, as shown. The generally tubular or cup-shaped fluid impermeable barrier 302 may at least partially define the outer surface of the sheath 344. The fluid impermeable barrier 302 may be similar or identical to and of the fluid impermeable barriers disclosed herein, in one or more aspects. For example, the fluid impermeable barrier 302 may be constructed of any of the materials disclosed herein for the fluid impermeable barrier. The fluid impermeable barrier 302 at least partially defines the chamber 304. For example, the inner surface of the fluid impermeable barrier 302 at least partially defines the perimeter of the chamber 304. The chamber 304 may at least temporarily retain bodily fluids therein. The fluid impermeable barrier 302 may also define an opening 306 extending through the fluid impermeable barrier 302 that is configured to have a male urethral opening positioned therethrough.

As shown, the fluid collection assembly 300 may include the porous material 310 therein. The porous material 310 may be similar or identical any of the porous materials disclosed herein, in one or more aspects. For example, the porous material 310 may include one or more of an outer layer 311 or and inner layer 312. The outer layer 311 may include an outer porous material.

The sheath 344 also includes at least a portion of the conduit 314 therein, such as at least partially disposed in the chamber 304. For example, the conduit 314 may extend from the sheath 344 at the distal end region 322 to a proximal end region 324 at least proximate to the opening 306. The proximal end region 324 may be disposed near or on the skin around the male urethral opening (e.g., on the penis or pubic area therearound). Accordingly, when an individual lays on their back, bodily fluids (e.g., urine) may aggregate near the opening 306 against the skin of the subject. The bodily fluids may be removed from the chamber 304 via the conduit 314.

In some examples, the fluid impermeable barrier 302 may be constructed of a material and/or have a thickness that allows the sheath 344 to collapse when placed under vacuum, such as to remove air around a penis in the fluid collection assembly 300 during use. In such examples, the conduit 314 may extend only to or into the distal end region 322 in the chamber 304 (e.g., not through to the area adjacent the opening 306). In such examples, urine may be collected and removed from the fluid collection assembly 300

In an example, portions of the chamber 304 may be substantially empty due to the varying sizes and rigidity of the male penis. However, in some examples, the outermost regions of the chamber 304 (e.g., periphery of the interior regions of the sheath 344) may include the porous material 310. For example, the porous material 310 may be bonded to the inner surface of the fluid impermeable barrier 302. The porous material 310 may be positioned (e.g., at the distal end of the chamber 304) to blunt a stream of urine from the male urethral opening thereby limiting splashing and/or to direct the bodily fluids to a selected region of the chamber 304. Since the chamber 304 is substantially empty (e.g., substantially all of the chamber 304 forms a reservoir), the bodily fluids are likely to pool at a gravimetrically low point of the chamber 304. The gravimetrically low point of the chamber 304 may be at an intersection of the skin of an individual and the fluid collection assembly 300, a corner formed in the sheath 344, or another suitable location depending on the orientation of the wearer.

The porous material 310 may include one or more of the outer layer 311 or the inner layer 312. The outer layer 311 and the inner layer 312 may be similar or identical to any of the fluid permeable membranes and the fluid permeable supports, respectively disclosed herein, in one or more aspects. One or more of the outer layer 311 or the inner layer 312 may be disposed between the fluid impermeable barrier 302 and a penis inserted into the chamber 304. The outer layer 311 may be positioned between the fluid impermeable barrier 302 and a penis inserted into the chamber 304, such as between the inner layer 312 and penis of a wearer as shown. The inner layer 312 may be positioned between the outer layer 311 and the fluid impermeable barrier 302. The inner surface of the fluid impermeable barrier 302, optionally including the end of the chamber 304 substantially opposite the opening 306, may be covered with one or both the outer layer 311 or the inner layer 312. The inner layer 312 or the outer layer 311 may be affixed (e.g., adhered) to the fluid impermeable barrier 302. The inner layer 312 or the outer layer 311 may be affixed to each other. In some examples, the porous material 310 only includes the outer layer 311 or the inner layer 312.

The fluid collection assembly 300 includes a cap 350 at a distal end region 322. The cap 350 defines an interior channel through which the bodily fluids may be removed from the fluid collection assembly 300. The interior channel is in fluid communication with the chamber 304. The cap 350 may be disposed over at least a portion of the distal end region 322 of one or more of the fluid impermeable barrier 302 or the porous material 310. The cap 350 may be made of a polymer, rubber, or any other fluid impermeable material. The cap 350 may be attached to one or more of the fluid impermeable barrier 302, the porous material 310, or the conduit 314. The cap 350 may cover at least a portion of the distal end region 322 of the fluid collection assembly 300. The cap 350 may define a fluid outlet 308 that is sized and configured to receive and fluidly seal against the conduit 314. The conduit 314 may extend a distance within or through the cap 350, such as to the porous material 310, through the porous material 310, or to a point set-off from the porous material 310. In an example, as depicted in FIG. 2B, the interior channel of the cap 350 may define a reservoir 320 therein.

The reservoir 320 is an unoccupied portion of device such as in the cap 350 and is void of other material. In some examples, the reservoir 320 is defined at least partially by the porous material 310 and the cap 350. During use, the bodily fluids that are in the chamber 304 may flow through the porous material 310 to the reservoir 320. The reservoir 320 may store at least some of the bodily fluids therein and/or position the bodily fluids for removal by the conduit 314. In some examples, at least a portion of the porous material 310 may extend continuously between at least a portion of the opening of the interior channel and chamber 304 to wick any bodily fluids from the opening directly to the reservoir 320.

In some examples (not shown), the fluid impermeable barrier 302 may be disposed on or over the cap 350, such as enclosing the cap 350 within the chamber 304.

In some examples, the sheath 344 may include at least a portion of the conduit 314 therein, such as at least partially disposed in the chamber 304. For example, the conduit 314 may extend from the sheath 344 to a region at least proximate to the opening 306. The inlet of the conduit 314 may be positioned adjacent to the annular base 352. The inlet of the conduit 314 may be positioned to be adjacent or proximate to the gravimetrically low point of the chamber 304, such as adjacent to the annular base 352. For example, the inlet may be co-extensive with or offset from the opening 306. In examples, the inlet may be positioned adjacent to the distal end region 322 of the sheath 344 (e.g., substantially opposite the opening 306).

The proximal end region 324 may be disposed near or on the skin around the male urethral opening (e.g., around the penis) and the inlet of the conduit 314 may be positioned in the proximal end region 324. The outlet of the conduit 314 may be directly or indirectly coupled to a vacuum source. Accordingly, bodily fluids may be removed from the proximal end region 324 of the chamber 304 via the conduit 314.

The base 342, the sheath 344, the cap 350, and the conduit 314 may be attached together using any suitable method. For example, at least two of the base 342, the sheath 344, the cap 350, or the conduit 314 may be attached together using at least one of an interference fit, an adhesive, stitching, welding (e.g., ultrasonic welding), tape, any other suitable method, or combinations thereof.

In some examples (not shown), the fluid collection assembly 300 may have a one piece design, with one or more of the sheath 344, the base 342, and the cap 350 being a single, integrally formed piece.

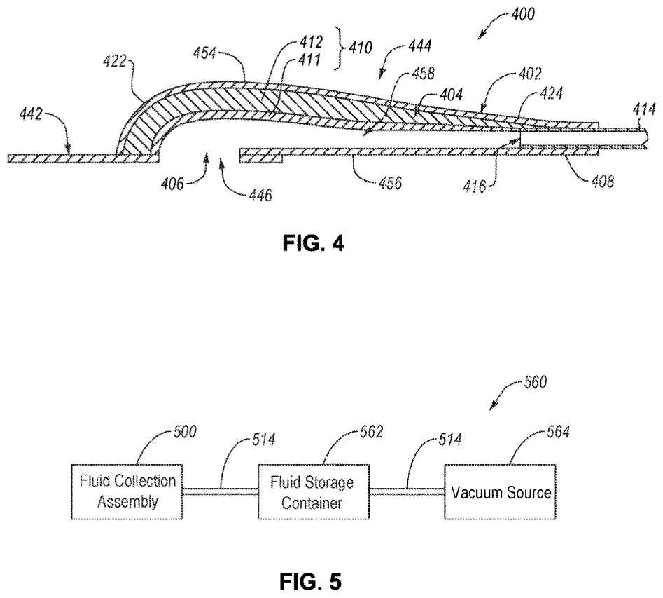

Also as shown, the conduit 314 may be at least partially disposed with the chamber of a fluid collection assembly. The conduit 314 may extend from the distal end region 322 to the proximal end region 324. For example, the conduit 314 may extend through the cap 350 to a point adjacent to the base 342. The conduit 314 is sized and positioned to be coupled to a fluid storage container or the vacuum source (FIG. 5). An outlet of the conduit 314 may be operably coupled to the vacuum source, directly or indirectly. The inlet 316 of the conduit 314 may be positioned within the chamber 304 such as at a location expected to be at the gravimetrically low point of the fluid collection assembly during use. By positioning the inlet 316 in a location expected to be at the gravimetrically low point of the fluid collection assembly when worn by the user, bodily fluids introduced into the chamber 304 may be removed via the conduit 314 to prevent pooling or stagnation of the bodily fluids within the chamber 304.

In some examples, the vacuum source may be remotely located from the fluid collection assembly 300. In such examples, the conduit 314 may be fluidly connected to the fluid storage container, which may be disposed between the vacuum source and the fluid collection assembly 300.

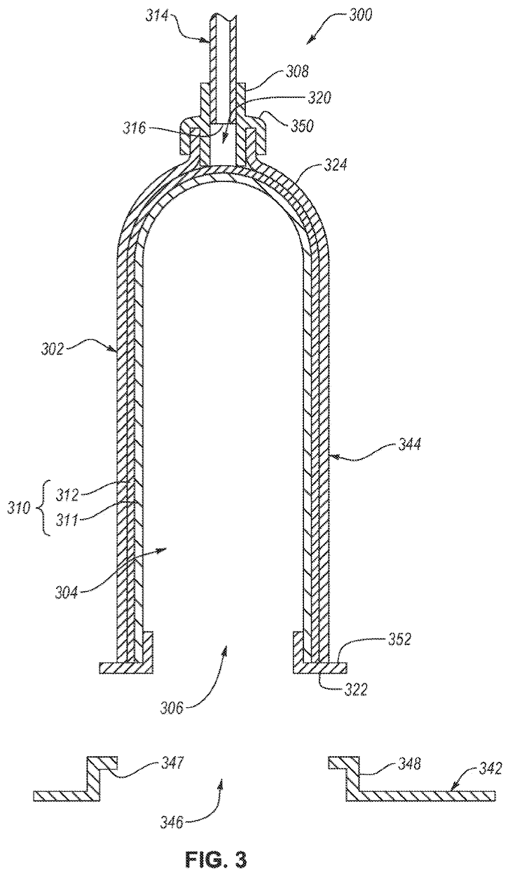

FIG. 4 is a cross-sectional schematic of a fluid collection assembly 400, according to an embodiment. The fluid collection assembly 400 is an example of a male fluid collection assembly though, in some embodiments, the fluid collection assembly 400 may be used to receive bodily fluids from a female urethral opening. Except as otherwise disclosed herein, the fluid collection assembly 400 is the same or substantially similar to any of the fluid collection assemblies disclosed herein. The sheath 444 includes a fluid impermeable barrier 402 that is at least partially formed from a first panel 454 and a second panel 456. The first panel 454 and the second panel 456 may be attached or integrally formed together (e.g., exhibits single piece construction). In an embodiment, as illustrated, the first panel 454 and the second panel 456 are distinct sheets. The fluid impermeable barrier 402 also defines a chamber 404 between the first panel 454 and the second panel 456, an opening 406 at a proximal end region 424 of the sheath 444, and a fluid outlet 408 at a distal end region 422 of the sheath 444. The sheath 444 also includes at least one porous material 410 disposed in the chamber 404.

The inner surface(s) of the fluid impermeable barrier 402 (e.g., inner surfaces of the first and second panels 454, 456 at least partially defines the chamber 404 within the fluid collection assembly 400. The fluid impermeable barrier 402 temporarily stores the bodily fluids in the chamber 404. The fluid impermeable barrier 402 may be formed from any of the fluid impermeable materials disclosed herein. As such, the fluid impermeable barrier 402 substantially prevents the bodily fluids from passing through the fluid impermeable barrier 402.

In an embodiment, at least one of the first panel 454 or the second panel 456 is formed from an at least partially transparent fluid impermeable material, such as polyethylene, polypropylene, polycarbonate, or polyvinyl chloride. Forming at least one of the first panel 454 or the second panel 456 from an at least partially transparent fluid impermeable material allows a person (e.g., medical practitioner) to examiner the penis. In some embodiments, both the first panel 454 and the second panel 456 are formed from at least partially transparent fluid impermeable material. Selecting at least one of the first panel 454 or the second panel 456 to be formed from an at least partially transparent impermeable material allows the penis to be examined without detaching the entire fluid collection assembly 400 from the region about the penis. For example, the chamber 404 may include a penis receiving area 458 that is configured to receive the penis of the individual when the penis extends into the chamber 404. The penis receiving area 458 may be defined by at least the porous material 410 and at least a portion of the at least partially transparent material of the first panel 454 and/or the second panel 456. In other words, the porous material 410 is positioned in the chamber 404 such that the porous material 410 is not positioned between the penis and at least a portion of the transparent portion of the first panel 454 and/or second panel 456 when the penis is inserted into the chamber 404 through the opening 406. The porous material 410 is generally not transparent and, thus, the portion of the at least partially transparent material of the first panel 454 and/or the second panel 456 that defines the penis receiving area 458 forms a window which allows the person to view into the penis receiving area 458 and examine the penis.

The fluid collection assembly 400 includes a sheath 444 and a base 442. The base 442 is configured to be attached (e.g., permanently attached to or configured to be permanently attached) to the sheath 444. The base 442 is also configured to be attached to the region about the urethral opening (e.g., penis) of the individual.

The opening 406 defined by the fluid impermeable barrier 402 provides an ingress route for bodily fluids to enter the chamber 404 when the penis is a buried penis and allow the penis to enter the chamber 404 (e.g., the penis receiving area 458) when the penis is not buried. The opening 406 may be defined by the fluid impermeable barrier 402 (e.g., an inner edge of the fluid impermeable barrier 402). For example, the opening 406 is formed in and extends through the fluid impermeable barrier 402 thereby enabling bodily fluids to enter the chamber 404 from outside of the fluid collection assembly 400.

The fluid impermeable barrier 402 defines the fluid outlet 408 sized to receive the conduit 414. The conduit 414 may be at least partially disposed in the chamber 404 or otherwise in fluid communication with the chamber 404 through the fluid outlet 408. The fluid outlet 408 may be sized and shaped to form an at least substantially fluid tight seal against the conduit 414 thereby substantially preventing the bodily fluids from escaping the chamber 404. In an embodiment, the fluid outlet 408 may be formed from a portion of the first panel 454 and the second panel 456 that are not attached or integrally formed together. In such an embodiment, the fluid impermeable barrier 402 may not include a cap exhibiting a rigidity that is greater than the portions of the fluid impermeable barrier 402 thereabout which may facilitate manufacturing of the fluid collection assembly 400 may decreasing the number of parts that are used to form the fluid collection assembly 400 and may decrease the time required to manufacture the fluid collection assembly 400. The lack of the cap may make securing the conduit 414 to the fluid outlet 408 using interference fit to be difficult though, it is noted, attaching the conduit 414 to the fluid outlet 408 may still be possible. As such, the conduit 414 may be attached to the fluid outlet 408 (e.g., to the first and second panels 454, 456) using an adhesive, a weld, or otherwise bonding the fluid outlet 408 to the fluid outlet 408. Attaching the conduit 414 to the fluid outlet 408 may prevent leaks and may prevent the conduit 414 from inadvertently becoming detached from the fluid outlet 408. In an example, the conduit 414 may be attached to the fluid outlet 408 in the same manufacturing step that attaches the first and second panels 454, 456 together. In an embodiment, the fluid collection assembly 400 includes a cap exhibiting a rigidity that is greater than portions of the fluid impermeable barrier 402 thereabout. In such an embodiment, the cap may form the fluid outlet 408. The cap may be selected to exhibit a maximum thickness that is less, than equal to, or slightly greater than (e.g., at most about 3 mm greater than) the thickness of the conduit 416 such that the cap has no significant effect on the ability of the fluid impermeable barrier 402 to lie flat.

As previously discussed, the sheath 444 includes at least one porous material 410 disclosed in the chamber 404. The porous material 410 may be the same or substantially similar to any of the porous materials disclosed herein in one or more aspects. For example, the porous material 410 may include an outer layer 411 including an outer porous material 411 and an inner layer 412. The porous material 410 may direct the bodily fluids to one or more selected regions of the chamber 404, such as away from the penis and towards the fluid outlet 408. The porous material 410 may be formed from any of the porous materials disclosed herein. In an example, the porous material 410 may be formed from a single layer, two layers, or three or more layers. In an example, the porous material 410 may be formed from a nonwoven material or a woven material (e.g., spun nylon fibers). In an example, the porous material 410 may include at least one material exhibiting substantially no absorption or at least one absorbent or adsorbent material.

In an embodiment, the porous material 410 may be a sheet. Forming the porous material 410 as a sheet may facilitate the manufacturing of the fluid collection assembly 400. For example, forming the porous material 410 as a sheet allows the first panel 454, the second panel 456, and the porous material 410 to each be sheets. During the manufacturing of the fluid collection assembly 400, the first panel 454, the second panel 456, and the porous material 410 may be stacked and then attached to each other in the same manufacturing step. For instance, the porous material 410 may exhibit a shape that is the same size or, more preferably, slightly smaller than the size of the first panel 454 and the second panel 456. As such, attaching the first panel 454 and the second panel 456 together along the outer edges thereof may also attach the porous material 410 to the first panel 454 and the second panel 456. The porous material 410 may be slightly smaller than the first panel 454 and the second panel 456 such that the first panel 454 and/or the second panel 456 extend around the porous material 410 such that the porous material 410 does not form a passageway through the fluid impermeable barrier 402 through which the bodily fluids may leak. Also, attaching the porous material 410 to the first panel 454 and/or the second panel 456 may prevent the porous material 410 from significantly moving in the chamber 404, such as preventing the porous material 410 from bunching together near the fluid outlet 408. In an example, the porous material 410 may be attached to the first panel 454 or the second panel 456 (e.g., via an adhesive) before or after attaching the first panel 454 to the second panel 456. In an example, the porous material 410 may merely be disposed in the chamber 404 without attaching the porous material 410 to at least one of the first panel 454 or the second panel 456. In an embodiment, the porous material 410 may exhibit shapes other than a sheet, such as a hollow generally cylindrical shape.

Generally, the sheath 444 is substantially flat when the penis is not in the penis receiving area 458 and the sheath 444 is resting on a flat surface. The sheath 444 is substantially flat because the fluid impermeable barrier 402 is formed from the first panel 454 and the second panel 456 instead of a generally tubular fluid impermeable barrier. Further, as previously discussed, the porous material 410 may be a sheet, which also causes the sheath 444 to be substantially flat. The sheath 444 may also be substantially flat because the fluid collection assembly 400 may not include relatively rigid rings or caps that exhibit a rigidity that is greater than the portions of the fluid impermeable barrier 402 thereabout since such rings and caps may inhibit the sheath 444 being substantially flat. It is noted that the sheath 444 is described as being substantially flat because at least one of the porous material 410 may cause a slight bulge to form in the sheath 444 depending on the thickness of the porous material 410, the fluid outlet 408 and/or conduit 414 may cause a bulge thereabout, or the base 442 may pull on portions of the sheath 444 thereabout. It is also noted that the sheath 444 may also be compliant and, as such, the sheath 444 may not be substantially flat during use since, during use, the sheath 444 may rest on a non-flat surface (e.g., may rest on the testicles, the perineum, and/or between the thighs) and the sheath 444 may conform to the surface of these shapes.

The ability of the sheath 444 to be substantially flat when the penis is not in the penis receiving area 458 and the sheath 444 is resting on a flat surface allows the fluid collection assembly 400 to be used with a buried and a non-buried penis. For example, when the fluid collection assembly 400 is being used with a buried penis, the penis does not extend into the penis receiving area 458 which causes the sheath 444 to lie relatively flat across the aperture 446 of the base 442. When the sheath 444 lies relatively flat across the aperture 446, the porous material 410 extends across the opening 406 and the aperature 446 and is in close proximity to the buried penis. As such, the porous material 410 prevents or inhibits pooling of bodily fluids discharged from the buried penis against the skin of the individual since the porous material 410 will receive and remove at least a significant portion of the bodily fluids that would otherwise pool against the skin of the individual. Thus, the skin of the individual remains dry thereby improving comfort of using the fluid collection assembly 400 and preventing skin degradation. However, unlike other conventional fluid collection assemblies that are configured to be used with buried penises, the fluid collection assembly 400 may still be used with a non-buried penis since the non-buried penis can still be received into the penis receiving area 458, even when the penis is fully erect. Additionally, the ability of the sheath 444 to be substantially flat allows the fluid collection assembly 400 to be used more discretely than if the sheath 444 was not substantially flat thereby avoiding possibly embarrassing scenarios.

When the sheath 444 is substantially flat, the porous material 410 occupies substantially all of the chamber 404 and the penis receiving area 458 is collapsed (shown as being non-collapsed in FIG. 4 for illustrative purposes to show the penis receiving area 458). In other words, the sheath 444 may not define a region that is constantly unoccupied by the porous material 410. When the porous material 410 occupies substantially all of the chamber 404, the bodily fluids discharged into the chamber 404 are unlikely to pool for significant periods of time since pooling of the bodily fluids may cause sanitation issues, cause an odor, and/or may cause the skin of the individual to remain in contact with the bodily fluids which may cause discomfort and skin degradation.

As previously discussed, the first panel 454, the second panel 456, and the porous material 410 may be selected to be relatively flexible. The first panel 454, the second panel 456, and the porous material 410 are relatively flexible when the first panel 454, the second panel 456, and the porous material 410, respectively, are unable to maintain their shape when unsupported. The flexibility of the first panel 454, the second panel 456, and the porous material 410 may allow the sheath 444 to be substantially flat, as discussed above. The flexibility of the first panel 454, the second panel 456, and the porous material 410 may also allow the sheath 444 to conform to the shape of the penis even when the size and shape of the penis changes (e.g., becomes erect) and to minimize any unoccupied spaces in the chamber 404 in which bodily fluids may pool.

As previously discussed, the fluid collection assembly 400 includes a base 442 that is configured to be attached to the sheath 444. For example, the base 442 is configured to be permanently attached to the sheath 444. The base 442 is configured to be permanently attached to the sheath 444 when, for example, when the fluid collection assembly 400 is provided with the base 442 permanently attached to the sheath 444 or the base 442 is provided without being permanently attached to the sheath 444 but is configured to be permanently attached to the sheath 444 at some point in the future. Permanently attached means that the sheath 444 cannot be detached from the base 442 without damaging at least one of the sheath 444 or the base 442, using a blade to separate the sheath 444 from the base 442, and/or using chemicals to dissolve the adhesive that attaches the sheath 444 from the base 442. The base 442 may be permanently attached to the sheath 444 using an adhesive, sewing, heat sealing, RF welding, or US welding. In an embodiment, the base 442 is configured to be reversibly attached to the sheath 444. In an embodiment, the base 442 is integrally formed with the sheath 444.

The base 442 includes an aperture 446. The base 442 is permanently attached to the distal end region 422 of the sheath 444 such that the aperture 446 is aligned with the opening 406.

The base 442 is sized, shaped, and made of a material to be coupled to the skin that surrounds the penis (e.g., mons pubis, thighs, testicles, and/or perineum) and have the penis disposed therethrough. For example, the base 442 may define an aperture 446 configured to have the penis positioned therethrough. In an example, the base 442 may exhibit the general shape or contours of the skin surface that the base 442 is configured to be coupled with. The base 442 may be flexible, thereby allowing the base 442 to conform to any shape of the skin surface and mitigate the base 442 pulling the on skin surface. The base 442 may extend laterally past the sheath 444 thereby increasing the surface area of the skin of the individual to which the fluid collection assembly 400 may be attached compared to a substantially similar fluid collection assembly 400 that did not include a base.

As previously discussed, the fluid collection assembly 400 includes the conduit 414. The inlet 416 of the conduit 414 may be located near the distal end region 422 of the sheath 444 which is expected to be the gravimetrically low point of the chamber 404 when worn by an individual. Locating the inlet 416 at or near the distal end region 422 of the sheath 444 enables the conduit 414 to receive more of the bodily fluids than if the inlet of the conduit 414 was located elsewhere and reduce the likelihood of pooling (e.g., polling of the bodily fluids may cause microbe growth and foul odors).

FIG. 5 is a block diagram of a fluid collection system 560 for fluid collection, according to an embodiment. The fluid collection system 560 includes a fluid collection assembly 500, a fluid storage container 562, and a vacuum source 564. The fluid collection assembly 500 may be the same or substantially similar to any of the fluid collection assemblies disclosed herein. The fluid collection assembly 500, the fluid storage container 562, and the vacuum source 564 may be fluidly coupled to each other via one or more conduits 514. For example, fluid collection assembly 500 may be operably coupled to one or more of the fluid storage container 562 or the vacuum source 564 via the conduit 514. The bodily fluids collected in the fluid collection assembly 500 may be removed from the fluid collection assembly 500 via the conduit 514 which protrudes into the fluid collection assembly 500. For example, an inlet of the conduit 514 may extend into the fluid collection assembly 500, such as to a reservoir therein. The outlet of the conduit 514 may extend into the fluid collection assembly 500 or the vacuum source 564. Suction force may be introduced into the chamber of the fluid collection assembly 500 via the inlet of the conduit 514 responsive to suction (e.g., vacuum) force applied at the outlet of the conduit 514.

The suction force may be applied to the outlet of the conduit 514 by the vacuum source 564 either directly or indirectly. The suction force may be applied indirectly via the fluid storage container 562. For example, the outlet of the conduit 514 may be disposed within the fluid storage container 562 and an additional conduit 514 may extend from the fluid storage container 562 to the vacuum source 564. Accordingly, the vacuum source 564 may apply suction to the fluid collection assembly 500 via the fluid storage container 562. The suction force may be applied directly via the vacuum source 564. For example, the outlet of the conduit 514 may be disposed within the vacuum source 564. An additional conduit 514 may extend from the vacuum source 564 to a point outside of the fluid collection assembly 500, such as to the fluid storage container 562. In such examples, the vacuum source 564 may be disposed between the fluid collection assembly 500 and the fluid storage container 562.

The fluid storage container 562 is sized and shaped to retain bodily fluids therein. The fluid storage container 562 may include a bag (e.g., drainage bag), a bottle or cup (e.g., collection jar), or any other enclosed container for storing bodily fluids such as urine. In some examples, the conduit 514 may extend from the fluid collection assembly 500 and attach to the fluid storage container 562 at a first point therein. An additional conduit 514 may attach to the fluid storage container 562 at a second point thereon and may extend and attach to the vacuum source 564. Accordingly, a vacuum (e.g., suction) may be drawn through fluid collection assembly 500 via the fluid storage container 562. Bodily fluids, such as urine, may be drained from the fluid collection assembly 500 using the vacuum source 564.

The vacuum source 564 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 564 may provide a vacuum or suction to remove bodily fluids from the fluid collection assembly 500. In some examples, the vacuum source 564 may be powered by one or more of a power cord (e.g., connected to a power socket), one or more batteries, or even manual power (e.g., a hand operated vacuum pump). In some examples, the vacuum source 564 may be sized and shaped to fit outside of, on, or within the fluid collection assembly 500. For example, the vacuum source 564 may include one or more miniaturized pumps or one or more micro pumps. The vacuum sources 564 disclosed herein may include one or more of a switch, a button, a plug, a remote, or any other device suitable to activate the vacuum source 564.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or +2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1. A fluid collection assembly, comprising:
a fluid impermeable barrier at least defining:
a chamber;
at least one opening; and
a fluid outlet; and
at least one porous material disposed in the chamber, the at least one porous material including an outer layer and an inner layer, the outer layer including at least one of bamboo, hydrophilic polypropylene, or hydrophilic polyethylene.

2. The fluid collection assembly of claim 1, wherein the outer layer includes bamboo.

3. The fluid collection assembly of claim 2, wherein the bamboo includes at least one of natural bamboo or black bamboo.

4. (canceled)

5. The fluid collection assembly of claim 2, wherein the outer layer includes a bamboo spunbonded nonwoven material.

6. The fluid collection assembly of claim 1, wherein outer layer material includes a woven material.

7. (canceled)

8. The fluid collection assembly of claim 1, wherein the outer layer includes a nonwoven material.

9. The fluid collection assembly of claim 1, wherein the outer layer includes a vertical nonwoven material.

10. The fluid collection assembly of claim 1, wherein the outer layer exhibits a thickness of about 1 mm or less.

11. The fluid collection assembly of claim 1, wherein the outer layer exhibits a thickness of about 25 μm to about 150 μm.

12. The fluid collection assembly of claim 1, wherein the outer layer exhibits a basis weight of about 25 g/m² to about 100 g/m².

13. (canceled)

14. The fluid collection assembly of any one of claim 1, wherein the inner layer includes a hydrophobic material.

15. The fluid collection assembly of any one of claim 1, wherein the inner layer includes at least one of a vertical nonwoven material or a porous foam.

16. The fluid collection assembly of claim 15, wherein the inner layer includes the porous foam, the porous foam including at least one of polyurethane, polyvinyl chloride, or polyethylene.

17. The fluid collection assembly of any one of claim 15, wherein the inner layer includes the porous foam, the porous foam exhibiting at least one of about 7 pores/cm² to about 13 pores/cm² or a density of about 16 kg/m³ to about 160 kg/m³.

18. (canceled)

19. The fluid collection assembly of claim 1, wherein the inner layer exhibits a thickness of about 8 mm to about 20 mm.

20. (canceled)

21. (canceled)

22. (canceled)

23. A fluid collection system, comprising:
a fluid collection assembly including:
a fluid impermeable barrier at least defining:
a chamber;
at least one opening; and
a fluid outlet; and
at least one porous material disposed in the chamber, the at least one porous material including an outer layer and an inner layer, the outer layer including at least one of bamboo, hydrophilic polypropylene, or hydrophilic polyethylene;
a fluid storage container; and
a vacuum source;
wherein the chamber of the fluid collection assembly, the fluid storage container, and the vacuum source are in fluid communication with each other such that, when one or more bodily fluids are present in the chamber, a suction provided from the vacuum source to the chamber of the fluid collection assembly removes the one or more bodily fluids from the chamber and deposits the bodily fluids in the fluid storage container.

24. A method of using a fluid collection system, the method comprising:
positioning a fluid collection assembly such that at least one opening defined by a fluid impermeable barrier of the fluid collection assembly is positioned adjacent to or receives a urethral opening, the fluid impermeable barrier of the fluid collection assembly at least defining a chamber and a fluid outlet, the fluid collection assembly including at least one porous material disposed in the chamber, the at least one porous material including an outer layer and an inner layer, the outer layer including at least one of a bamboo, hydrophilic polypropylene, or hydrophilic polyethylene; and
receiving one or more bodily fluids from the urethral opening into the at least one porous material.

25. The fluid collection assembly of claim 1, wherein the outer layer includes hydrophilic polypropylene.

26. The fluid collection assembly of claim 1, wherein the outer layer includes two or more of bamboo, hydrophilic polypropylene, or hydrophilic polyethylene.

27. The fluid collection assembly of claim 1, wherein the outer layer includes bamboo and hydrophilic polypropylene.

28. The fluid collection assembly of claim 1, wherein the at least one porous material further includes cellulose or hydrophilic polyester.

29. The fluid collection assembly of claim 1, wherein the inner layer includes polyester.

30. The fluid collection assembly of claim 1, wherein the at least one porous material includes at least one additional layer.
